(19) United States
(12) Patent Application Publication
(10) Pub. No.: US 2025/0040503 A1
(43) Pub. Date: Feb. 6, 2025

(54) METHOD FOR PRODUCING HAPLOID, AND DOUBLED HAPLOID BETA VULGARIS PLANTS BY MICROSPORE CULTURE

(71) Applicant: KWS SAAT SE & Co. KGaA, Einbeck (DE)

(72) Inventor: Clemens SPRINGMANN, Bad Gandersheim (DE)

(73) Assignee: KWS SAAT SE & Co. KGaA, Einbeck (DE)

(21) Appl. No.: 18/718,641

(22) PCT Filed: Dec. 22, 2022
(86) PCT No.: PCT/EP2022/087525

§ 371 Date: Jun. 11, 2024

Related U.S. Application Data
(60) Provisional application No. 63/292,564, filed on Dec. 22, 2021.

(30) Foreign Application Priority Data
May 10, 2022 (EP) 22172521.1

(51) Int. Cl. A01H 4/00; A01H 6/02

(52) U.S. Cl. CPC A01H 4/008; A01H 4/002; A01H 4/005; A01H 6/024

(57) ABSTRACT

The present invention relates to a method for the production of haploid, polyhaploid and/or doubled haploid embryos, calli, seeds and/or plants of the species Beta vulgaris from isolated microspore cultures, more specifically to a method comprising contacting the isolated microspores with a histone deacetylase inhibitor (HDACi) and a complex protein composition. The present invention also provides a kit for producing a haploid, polyhaploid and/or doubled haploid embryo, callus, seed and/or plant of the species Beta vulgaris from at least one isolated microspore as well as the use of a histone deacetylase inhibitor (HDACi) and a complex protein composition for producing a haploid, polyhaploid and/or doubled haploid embryo, callus, seed and/or plant of the species Beta vulgaris. Finally, the present invention also relates to a population of haploid, polyhaploid and/or doubled haploid Beta vulgaris plants directly derived from a single flower, single inflorescence or single bud.

### TECHNICAL FIELD

The present invention relates to a method for the production of haploid, polyhaploid and/or doubled haploid embryos, calli, seeds and/or plants of the species Beta vulgaris from isolated microspore cultures, especially in a non-genotype dependent way, more specifically to a method comprising contacting the isolated microspores with a histone deacetylase inhibitor (HDACi) and a complex protein composition. The present invention also provides a kit for producing a haploid, polyhaploid and/or doubled haploid embryo, callus, seed and/or plant of the species Beta vulgaris from at least one isolated microspore as well as the use of a histone deacetylase inhibitor (HDACi) and a complex protein composition for producing a haploid, polyhaploid and/or doubled haploid embryo, callus, seed and/or plant of the species Beta vulgaris. Finally, the present invention also relates to a population of haploid, polyhaploid and/or doubled haploid Beta vulgaris plants directly derived from a single flower, single inflorescence or single bud.

### BACKGROUND

Subspecies of Beta vulgaris are valuable vegetables (red beet and swiss chard), feed (fodder beet) and technical (sugar beet) crops. Currently, the main trend in the breeding of these crops is development of F1 hybrids, based on the crossing of two homozygous parental lines. In conventional breeding, plant homozygosity is obtained after several cycles of inbreeding by self-pollination followed by selection of phenotypically uniform families over a minimum of 4-6 generations which takes 8 to 12 years for biennial crops. Further, this process does not ensure complete homozygosity in case of allogamous species like Beta vulgaris.

The long-term development of parental lines by inbreeding is one of the limiting factors and a major problem in competitive F1 hybrids breeding. Doubled haploids (OH) technologies offer a time-saving approach to obtain pure breeding lines by reducing the period to approximately 3-5 years (Zhuzhzhalova, T. P., et al. “Biotechnological methods as a tool for efficient sugar beet breeding.” Vavilov Journal of Genetics and Breeding 24.1 (2020): 40.). Advantages of the doubled haploid production are the ability to achieve fully homozygous plant genotypes in one generation as well as the manifestation of recessive alleles in haploid plants usually masked by the heterozygous state of a diploid plant, whereby the identification, assessment, and selection of plants with traits of agronomic importance is facilitated.

Haploid plants can occur spontaneously or develop as the result of apomixis and chromosome elimination after interspecific or intergeneric hybridization. Additionally haploid plants may be generated after induction of gametogenesis in microspore, anther, ovule or ovary culture.

In sugar beet, however, even at date most of these methods fail or are inefficient. Microspore culture led to induction of only callus or proembryo structures without further regeneration, or somatic clones (Grigolava, T. R., et al. “Methodological approaches for producing doubled haploids in sugar beet and red beet (Beta vulgaris L.).” Vavilov Journal of Genetics and Breeding 25.3 (2021): 276.). Callus, roots or plants obtained in culture were diploid and their gametophytic origin could not be confirmed. Early attempts to produce sugar beet haploids in anthers culture did not lead to obtaining androgenic plants. In 2017 Gorecka et al. managed to obtain embryos of red beet in microspores and anthers culture, however, the unrooted rosettes obtained from them died (Gorecka, Krystyna, et al. “Development of embryoids by microspore and anther cultures of red beet (Beta vulgaris L. subsp. vulgaris).” Journal of Central European Agriculture 18.1 (2017): 185-195.).

So far, exclusively gynogenesis has been reported as the only way for successful production of Beta vulgaris haploid and OH plants. Unfertilized ovules are excised from ovaries of male sterile or fertile donor plants and cultured in vitro. Gynogenic embryos originating from the egg cell convert into shoots with a haploid chromosome number. A single set of chromosomes in haploids is doubled by treating shoot meristems with anti-mitotic agents or by culturing the shoots on media supplemented with such compounds. This results in the development of doubled haploid (OH) shoots that are completely homozygous.

Even though production of haploid plants from gynogenesis has been implemented in sugar beet breeding programs for the development of OH lines, gynogenesis is highly laborious and expensive. Microspore cultures to develop haploids in Beta spp. are presently not available.

There is thus still a great need to increase efficiency in production of haploid and double haploid Beta vulgaris plants.

### SUMMARY OF THE INVENTION

As specified in the Background Section, there is a great need in the art to identify technologies for efficient production of haploid and double haploid Beta vulgaris plants and use this understanding to develop novel methods and kits for producing such plants. The present invention satisfies this and other needs.

In one aspect, the present invention relates to a method for producing a haploid, polyhaploid and/or doubled haploid embryo, callus, seed and/or plant of the species Beta vulgaris comprising:
i) providing at least one isolated microspore of Beta vulgaris,
ii) culturing the at least one microspore from step i) in the presence of a complex protein composition and at least one histone deacetylase inhibitor (HDACi),
iii) culturing the at least one microspore from step ii) to obtain a callus or an embryo, and
iv) optionally, regenerating and thereby obtaining at least one plant from the callus or embryo of step iii), and preferably obtaining at least one haploid, polyhaploid and/or doubled haploid seed thereof.

In one embodiment of the method described above, step i) comprises one or more of the following steps:
(a) providing a bud or an inflorescence or a part thereof comprising microspores at the tetrad and uninuclear development stage,
(b) optionally, disinfecting the bud or an inflorescence or the part thereof of (a),
(c) transferring the bud or an inflorescence or the part thereof of (a) or (b) to medium containing macro- and microsalts, saccharides and a complex protein composition,
(d) homogenizing the medium containing the bud or an inflorescence or the part thereof of step (c) and thereby producing a cell suspension,
(e) optionally, purifying the cell suspension preferably by means of a strainer and/or centrifugation thereby
(f) producing a suspension of microspores with a density of around 10,000 to 500,000 microspores per ml medium,
(g) optionally, cultivating the microspore suspension in the dark, and
(h) adding at least one histone deacetylase inhibitor (HDACi) to a final concentration of 1 nM to 10 μM, preferably 100 nM to 10 μM, more preferably 1 μM to 10 μM.

In a further embodiment of the method described above, step i) comprises all steps (a)-(h).

In another embodiment of the method according to any of the embodiments described above, the step ii) of the method further comprises one or more of the following steps:
(i) optionally, cultivating the microspore suspension in the dark,
(j) adding at least one histone deacetylase inhibitor (HDACi) to a final concentration of 1 nM to 10 μM, preferably 100 nM to 10 μM, more preferably 1 μM to 10 μM and
(k) removing the at least one histone deacetylase inhibitor (HDACi) from the microspore culture of step ii).

In one embodiment of the method described above, step ii) comprises all steps (i)-(k). In a further embodiment of the method according to any of the embodiments described above, the at least one histone deacetylase inhibitor (HDACi) is selected from the group consisting of trichostatin A (TSA), hydroxamic acids and hydroxamates, such as vorinostat (SAHA), belinostat (PXD101), dacinostat (LAQ824), and panobinostat (LBH589), cyclic tetrapeptides, such as trapoxin B and depsipeptides, such as romidepsin (FK228), benzamides such as entinostat (MS-275), tacedinaline (C1994), and mocetinostat (MGCD0103), electrophilic ketones, and aliphatic acid compounds such as phenylbutyrate and valproic acid, preferably wherein the histone deacetylase inhibitor (HDACi) is trichostatin A (TSA) or romidepsin (FK228).

In another embodiment of the method according to any of the embodiments described above, the complex protein composition comprises or consists of hydrolysed or partially hydrolysed protein matter derived from milk, such as casein or whey, animals, such as meat or fish, cereal, such as rice or corn, plants, such as soybean or combinations thereof and/or the complex protein composition comprises or consists of hydrolysed milk protein isolates, hydrolysed lactoprotein concentrate, hydrolysed casein isolates, casein hydrolysates, hydrolysed lactalbumin, hydrolysed casein sodium, hydrolysed calcium caseinate, hydrolysed full cow's milk, partially or completely skimmed milk, hydrolysed soy protein isolate, hydrolysed soybean concentrate or combinations thereof and/or the complex protein composition comprises or consists of a proteolysate selected from the group consisting of casein hydrolysate, soybean hydrolysate, rice proteolysate, potato protein hydrolysate, fish protein hydrolysate, ovalbumin hydrolysate, lactalbumin hydrolysate, glutin hydrolysate, animal and plant proteolysate and a combination thereof, preferably the hydrolysis degree is in a range from about 20 to about 80%, preferably from about 30 to about 80%, particularly preferably from about 40 to about 60%.

In one embodiment of the method according to any of the embodiments described above, the at least one histone deacetylase inhibitor (HDACi) is present at a concentration from about 1 nM to about 10 μM, preferably 100 nM to 10 μM, more preferably 1 μM to 10 μM in the culture medium used in step ii) and/or the complex protein composition is present at a concentration from about 100 to about 20,000 mg/l, preferably about 500 to about 15,000 mg/l, particularly preferably about 2,000 to about 10,000 mg/l in the culture medium used in step ii).

In another embodiment of the method according to any of the embodiments described above, the callus or embryo is contacted with one or more plant growth regulator(s) selected from auxins, cytokinins, gibberellins, abscisic acid and mixtures thereof in step iv). Preferably, in step iv) the callus or embryo is contacted with at least gibberellic acid such as GA3 and/or thidiazuron (TDZ), for example both in a concentration of about 1 mg/l.

In a further embodiment of the method according to any of the embodiments described above, one or more chromosome doubling agent(s), such as colchicine, is/are added during step iii) and/or step iv).

In another aspect, the present invention relates to a kit for producing a haploid, polyhaploid and/or doubled haploid embryo, callus, seed and/or plant of the species Beta vulgaris from at least one isolated microspore comprising:
(a) at least one histone deacetylase inhibitor (HDACi), and
(b) a complex protein composition,

wherein the at least one histone deacetylase inhibitor (HDACi) and the complex protein composition are comprised within the same container or within two or more separate containers.

In one embodiment of the kit described above, the at least one histone deacetylase inhibitor (HDACi) is selected from the group consisting of trichostatin A (TSA), hydroxamic acids and hydroxamates, such as vorinostat (SAHA), belinostat (PXD101), dacinostat (LAQ824), and panobinostat (LBH589), cyclic tetrapeptides, such as trapoxin B and depsipeptides, such as romidepsin (FK228), benzamides such as entinostat (MS-275), tacedinaline (C1994), and mocetinostat (MGCD0103), electrophilic ketones, and aliphatic acid compounds such as phenylbutyrate and valproic acid, preferably the histone deacetylase inhibitor (HDACi) is trichostatin A (TSA) or romidepsin (FK228).

In another embodiment of the kit according to any of the embodiments described above, the complex protein composition comprises or consists of hydrolysed or partially hydrolysed protein matter derived from milk, such as casein or whey, animals, such as meat or fish, cereal, such as rice or corn, plants, such as soybean or combinations thereof and/or the complex protein composition comprises or consists of hydrolysed milk protein isolates, hydrolysed lactoprotein concentrate, hydrolysed casein isolates, casein hydrolysates, hydrolysed lactalbumin, hydrolysed casein sodium, hydrolysed calcium caseinate, hydrolysed full cow's milk, partially or completely skimmed milk, hydrolysed soy protein isolate, hydrolysed soybean concentrate or combinations thereof and/or the complex protein composition comprises or consists of a proteolysate selected from the group consisting of casein hydrolysate, soybean hydrolysate, rice proteolysate, potato protein hydrolysate, fish protein hydrolysate, ovalbumin hydrolysate, lactalbumin hydrolysate, glutin hydrolysate, animal and plant proteolysate and a combination thereof, preferably the hydrolysis degree is in a range from 20 to 80%, preferably 30 to 80%, particularly preferably 40 to 60%.

In another embodiment of the kit according to any of the embodiments described above, the kit further comprises one or more plant growth regulator(s) selected from auxins, cytokinins, gibberellins, abscisic acid, and mixtures thereof and/or the kit further comprises one or more chromosome doubling agent(s) such as colchicine.

In another aspect, the present invention relates to the use of a histone deacetylase inhibitor (HDACi), preferably as defined above and a complex protein composition, preferably as defined above, or the use of a kit as defined in any of the embodiments described above for producing a haploid, polyhaploid and/or doubled haploid embryo, callus and/or plant or seed of the species Beta vulgaris, preferably in a method according to any of the embodiments described above.

In yet another aspect, the present invention relates to a population of haploid, polyhaploid and/or doubled haploid Beta vulgaris plants directly derived from a single flower, single inflorescence or single bud, preferably obtained or obtainable by a method according to any of the embodiments described above.

In one embodiment of the population described above, the population comprises at least individuals.

### Definitions

Androgenesis is defined as the process of generation of an individual whose genetic background is derived exclusively from a nucleus of male origin. That is, androgenesis is the generation of a plant exclusively from a male, haploid gamete precursor (gametophyte).

Haploid is an attribute applicable to cells or to plants or parts of plants, of which the chromosomes contained in their nucleus are each in only one copy (n).

Diploid is an attribute applicable to cells or to plants or parts of plants, of which the chromosomes contained in their nucleus are each in two copies (2n).

Doubled haploid is an attribute applicable to cells or to plants or parts of plants comprising said cells, the chromosome stock of which was multiplied artificially, most often by chemical treatment, such as with colchicine, or by spontaneous doubling. This doubling of the chromosome stock makes it possible to obtain a cell, plant or plant part that has two copies of each chromosome in its nucleus (2n), wherein said cell, plant or plant part is entirely homozygous or essentially homozygous.

Polyhaploid is an attribute applicable to cells or to plants or parts of plants comprising said cells, these cells being haploid initially, and their chromosome stock having tripled or more spontaneously. The cell, plant or plant part that has at least three copies of each chromosome in its nucleus (3n or 4n, etc.), wherein said cell, plant or plant part is entirely homozygous or essentially homozygous.

The term microspore is herein used to designate an immature male gametophyte of a plant at all stages of its in vitro growth, including its multicellular form derived from the sporophytic divisions of a single cell isolated microspore, and still enclosed within the original exine wall (this multicellular form is herein also referred to as a multicellular structure). At the uninucleate development stage of microspores, the nucleus is not divided yet. A tetrad refers to microspores arranged in a group of four after a diploid cell undergoes meiosis to form four haploid microspores.

A plant of the species Beta vulgaris is, in particular, a plant of the subspecies Beta vulgaris subsp. vulgaris. For example, among these are Beta vulgaris subsp. vulgaris var. altissima (sugar beet in a narrower sense), Beta vulgaris ssp. vulgaris var. vulgaris (swiss chard), Beta vulgaris ssp. vulgaris var. conditiva (beetroot/red beet), Beta vulgaris ssp. vulgaris var. crassa/alba (fodder beet).

A complex protein composition in the context of the present disclosure refers to a mixture of proteins derived from milk, meat, fish, cereal or soybeans. Preferably, the complex protein composition comprises or consist of at least partially hydrolysed proteins. In particular, a complex protein composition may be a proteolysate, i.e. a protein mixture obtained by enzymatic hydrolysis catalyzed by proteases. The hydrolysis degree is the percentage of peptide bonds cleaved with respect to the total number of bonds available for proteolytic hydrolysis. Examples for complex protein compositions are given in the detailed description.

Histone deacetylases are enzymes, which remove acetyl groups from acetyl lysine at the N-terminus of histones thereby increasing the affinity of the histone to DNA. Histone deacetylases therefore play a role in the regulation of DNA expression. Inhibitors of histone deacetylases (HDACi) inhibit histone deacetylases and therefore prevent the removal of acetyl groups from the histones. Histone deacetylase inhibitors are used in the treatment of several diseases.

A bud refers to an embryonic shoot occurring in the axil of a leaf or at the tip of a stem. A bud may be specialized to develop flowers or inflorescence.

Macro salts may be selected from the group consisting of ammonium nitrate (NH₄NO₃), calcium chloride (CaCl₂×2H₂O), magnesium sulfate (MgSO₄×7H₂O), monopotassium phosphate (KH₂PO₄), dipotassium phosphate (K₂HPO₄), potassium nitrate (KNO₃), calcium nitrate (Ca(NO₃)₂×4H₂O); micro salts may be selected from boric acid (H₃BO₃), cobalt chloride (COCl₂×6H₂O), ferrous sulfate (FeSO₄×7H₂O), manganese(II) sulfate (MnSO₄×4H₂O), potassium iodide (KI), sodium molybdate (Na₂MoO₄×2H₂O), zinc sulfate (ZnSO₄×7H₂O), ethylenediaminetetraacetic acid ferric sodium (FeNaEDTA), copper sulfate (CuSO₄×5H₂O).

Plant growth regulators are chemical compounds, which affect the growth and development of plants, e.g. by promoting or inhibiting growth. Natural plant growth regulators are plant hormones, which plants produce themselves. Synthetic plant growth regulators, on the other hand, do not naturally occur in plants. Examples of plant growth regulators are given in the detailed description.

Haploid plants can undergo spontaneous chromosome doubling or chromosome doubling can be enhanced or facilitated by a chromosome doubling agent, which e.g. blocks the function of the spindle fibers during meiosis or mitosis. The most commonly used chromosome doubling agent is colchicine.

### BRIEF DESCRIPTION OF THE DRAWINGS

FIG. 1 (FIG. 1.) shows the development of sugar beet microspores to callus structures (panel A1: symmetrical division of the microspore nucleus; panel A2: 4 nuclei stage; panels A3-A6: multinuclear cells and callus clusters).

FIG. 2 (FIG. 2) shows the development of sugar beet microspores into callus clusters.

FIG. 3 (FIG. 3) shows shoot induction from microspore-derived sugar beet calli.

FIG. 4 (FIG. 4) shows a comparison of the callus induction potential of SAHA vs. TSA. DR=monogerm (cf. Examples below). 1 SAHA=1 μM; 5 SAHA=5 μM. TSA was used in a concentration of 5 μM in this example.

FIG. 5 (FIG. 5) shows a comparison of the callus induction potential of TSA and romidepsin as well as the combined effect of both HDACis. DR=monogerm (cf. Examples below). Romidepsin (alone or in combination with TSA) was used in a concentration of 2.5 μM. TSA was used in a concentration of 5 μM in this example.

FIG. 6 (FIG. 6) shows a comparison of the callus induction potential of TSA and CPI-203, a bromodomain inhibitor of targetmol. CPI was used in concentrations of 1 μM; 2.5 μM and 5 μM and had a very small, but positive, effect on callus induction. TSA was again used as control for the six genotypes tested. TSA was used in a concentration of 5 μM in this example.

FIG. 7 (FIG. 7) shows a comparison of the callus induction potential of TSA and I-Bet 726, another bromodomain inhibitor. I-Bet was used in concentrations of 1 μM, 2.5 μM and 5 μM in comparison to TSA. TSA was used in a concentration of 5 μM in this example.

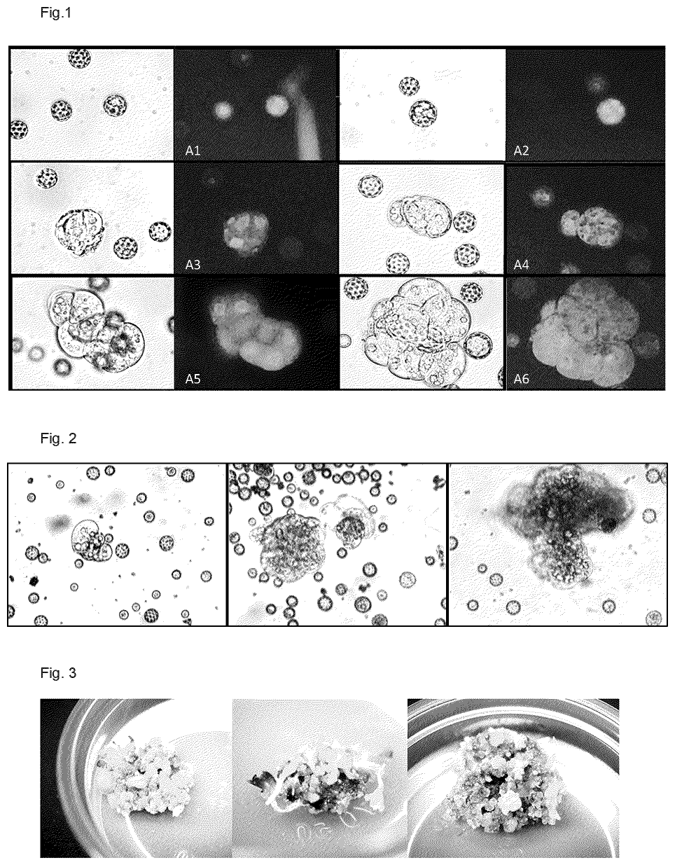

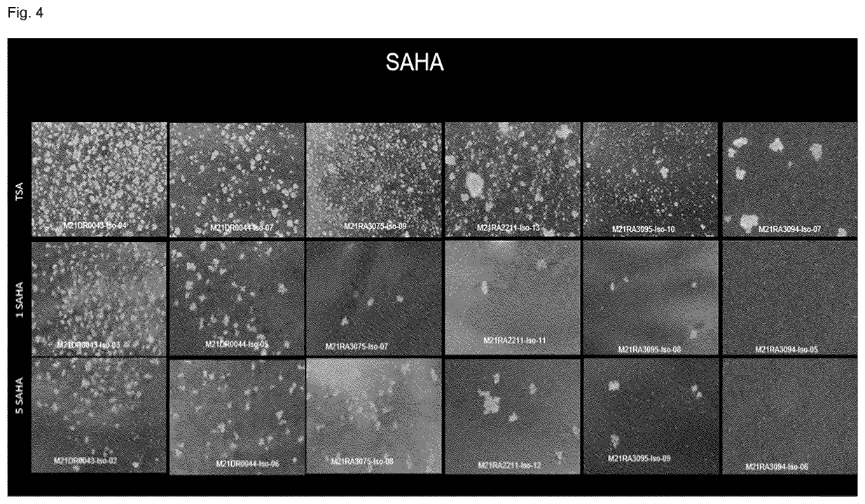

Remark concerning the data shown in FIG. 4 to FIG. 7:

The experimental set-up is detailed in the below Examples. In the Figures, each column represents a different genotype. Each row shows a different treatment (agent indicated on the left before each row). Time point, duration and buffer used were always identical for all treatments in the different rows. The different rows thus only differ from each other in the kind of inhibitor used.

All pictures were taken with a ZEISS Stemi 2000-C microscope using a 10-times magnification.

### DETAILED DESCRIPTION

As specified in the Background Section, there is a great need in the art to identify technologies for efficient production of haploid and double haploid Beta vulgaris plants and use this understanding to develop novel methods and kits for producing such plants. The present invention satisfies this and other needs. Embodiments of the present invention relate generally to methods and kits for the production of haploid, polyhaploid and/or doubled haploid Beta vulgaris plants by isolated microspore cultures, especially in a genotype independent way, and more specifically to methods and kits for producing such Beta vulgaris plants comprising contacting the isolated microspores with an inhibitor of histone deacetylase (HDACi) and a complex protein composition.

To facilitate an understanding of the principles and features of the various embodiments of the invention, various illustrative embodiments are explained below. Although exemplary embodiments of the invention are explained in detail, it is to be understood that other embodiments are contemplated. Accordingly, it is not intended that the invention is limited in its scope to the details of construction and arrangement of components set forth in the following description or examples.

The invention is capable of other embodiments and of being practiced or carried out in various ways. Also, in describing the exemplary embodiments, specific terminology will be resorted to for the sake of clarity.

It must also be noted that, as used in the specification and the appended claims, the singular forms “a,” “an” and “the” include plural references unless the context clearly dictates otherwise. For example, reference to a component is intended also to include composition of a plurality of components. References to a composition containing “a” constituent is intended to include other constituents in addition to the one named. In other words, the terms “a,” “an,” and “the” do not denote a limitation of quantity, but rather denote the presence of “at least one” of the referenced item.

As used herein, the term “and/or” may mean “and,” it may mean “or,” it may mean “exclusive-or,” it may mean “one,” it may mean “some, but not all,” it may mean “neither,” and/or it may mean “both.” The term “or” is intended to mean an inclusive “or.”

Also, in describing the exemplary embodiments, terminology will be resorted to for the sake of clarity. It is intended that each term contemplates its broadest meaning as understood by those skilled in the art and includes all technical equivalents which operate in a similar manner to accomplish a similar purpose. It is to be understood that embodiments of the disclosed technology may be practiced without these specific details. In other instances, well-known methods, structures, and techniques have not been shown in detail in order not to obscure an understanding of this description. References to “one embodiment,” “an embodiment,” “example embodiment,” “some embodiments,” “certain embodiments,” “various embodiments,” etc., indicate that the embodiment(s) of the disclosed technology so described may include a particular feature, structure, or characteristic, but not every embodiment necessarily includes the particular feature, structure, or characteristic.

Further, repeated use of the phrase “in one embodiment” does not necessarily refer to the same embodiment, although it may.

As used herein, the term “about” should be construed to refer to both of the numbers specified as the endpoint(s) of any range. Any reference to a range should be considered as providing support for any subset within that range. Ranges may be expressed herein as from “about” or “approximately” or “substantially” one particular value and/or to “about” or “approximately” or “substantially” another particular value. When such a range is expressed, other exemplary embodiments include from the one particular value and/or to the other particular value. Further, the term “about” means within an acceptable error range for the particular value as determined by one of ordinary skill in the art, which will depend in part on how the value is measured or determined, i.e., the limitations of the measurement system. For example, “about” can mean within an acceptable standard deviation, per the practice in the art. Alternatively, “about” can mean a range of up to ±20%, preferably up to ±10%, more preferably up to ±5%, and more preferably still up to ±1% of a given value. Alternatively, particularly with respect to biological systems or processes, the term can mean within an order of magnitude, preferably within 2-fold, of a value. Where particular values are described in the application and claims, unless otherwise stated, the term “about” is implicit and in this context means within an acceptable error range for the particular value.

Throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

Similarly, as used herein, “substantially free” of something, or “substantially pure”, and like characterizations, can include both being “at least substantially free” of something, or “at least substantially pure”, and being “completely free” of something, or “completely pure”.

By “comprising” or “containing” or “including” is meant that at least the named compound, element, particle, or method step is present in the composition or article or method, but does not exclude the presence of other compounds, materials, particles, method steps, even if the other such compounds, material, particles, method steps have the same function as what is named.

Throughout this description, various components may be identified having specific values or parameters, however, these items are provided as exemplary embodiments. Indeed, the exemplary embodiments do not limit the various aspects and concepts of the present invention as many comparable parameters, sizes, ranges, and/or values may be implemented. The terms “first,” “second,” and the like, “primary,” “secondary,” and the like, do not denote any order, quantity, or importance, but rather are used to distinguish one element from another.

It is noted that terms like “specifically,” “preferably,” “typically,” “generally,” and “often” are not utilized herein to limit the scope of the claimed invention or to imply that certain features are critical, essential, or even important to the structure or function of the claimed invention. Rather, these terms are merely intended to highlight alternative or additional features that may or may not be utilized in a particular embodiment of the present invention. It is also noted that terms like “substantially” and “about” are utilized herein to represent the inherent degree of uncertainty that may be attributed to any quantitative comparison, value, measurement, or other representation.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “50 mm” is intended to mean “about 50 mm.”

It is also to be understood that the mention of one or more method steps does not preclude the presence of additional method steps or intervening method steps between those steps expressly identified. Similarly, it is also to be understood that the mention of one or more components in a composition does not preclude the presence of additional components than those expressly identified.

The materials described hereinafter as making up the various elements of the present invention are intended to be illustrative and not restrictive. Many suitable materials that would perform the same or a similar function as the materials described herein are intended to be embraced within the scope of the invention. Such other materials not described herein can include, but are not limited to, materials that are developed after the time of the development of the invention, for example. Any dimensions listed in the various drawings are for illustrative purposes only and are not intended to be limiting. Other dimensions and proportions are contemplated and intended to be included within the scope of the invention.

In accordance with the present invention there may be employed conventional molecular biology, microbiology, and recombinant DNA techniques within the skill of the art. Such techniques are explained fully in the literature. See, e.g., Sambrook, Fritsch & Maniatis, Molecular Cloning: A Laboratory Manual, Second Edition (1989) Cold Spring Harbor Laboratory Press, Cold Spring Harbor, New York (herein “Sambrook et al., 1989”); DNA Cloning: A Practical Approach, Volumes I and II (D. N. Glover ed. 1985); Oligonucleotide Synthesis (M. J. Gait ed. 1984); Nucleic Acid Hybridization (B. D. Hames & S. J. Higgins eds. (1985); Transcription and Translation (B. D. Hames & S. J. Higgins, eds. (1984); Animal Cell Culture (R. I. Freshney, ed. (1986); Immobilized Cells and Enzymes (IRL Press, (1986); B. Perbal, A Practical Guide To Molecular Cloning (1984); F. M. Ausubel et al. (eds.), Current Protocols in Molecular Biology, John Wiley & Sons, Inc. (1994); among others.

### Methods and Kits of the Invention

In this context, one of the aims of the present invention is to provide a method for the production of haploid, polyhaploid and/or doubled haploid Beta vulgaris plants by or from isolated microspore cultures, especially in a non-genotype dependent (i.e., genotype independent) way.

Another aim of the invention is to provide calli, embryos, plantlets and plants regenerated from the embryo, progeny of such plants and seed from such plants, usable in or obtained by or obtainable by a method for the production of haploid, polyhaploid and/or doubled haploid Beta vulgaris plants by or from isolated microspore cultures, especially in a non-genotype dependent way.

Such aims are achieved by the present invention, which relates, in one aspect, to a method for producing a haploid, polyhaploid and/or doubled haploid embryo, callus, seed and/or plant of the species Beta vulgaris comprising:
i) providing at least one isolated microspore of Beta vulgaris,
ii) culturing the at least one microspore from step i) in the presence of a complex protein composition and at least one histone deacetylase inhibitor (HDACi),
iii) culturing the at least one microspore from step ii) to obtain a callus or an embryo, and
iv) optionally, regenerating and thereby obtaining at least one plant from the callus or embryo of step iii), and preferably obtaining at least one haploid, polyhaploid and/or doubled haploid seed thereof.

The method according to the present invention provides the advantage, that hundreds of haploid or doubled haploid plants can be obtained from one flower, inflorescence or bud while, using ovule culture, a maximum four plants can be obtained from one flower, inflorescence or bud. A further advantage is that haploid plants obtained by microspore culture have a much higher spontaneous doubling rate than those obtained from ovule culture. Plants obtained from ovule culture show a maximum of 5% spontaneous doubling, while those obtained from microspore culture show frequently spontaneous doubling rates of over 50%. As such, the methods of the present invention are no essentially biological processes for the production of plants in view of the inherent use of microspore culture performed in the presence of a HDACi and the subsequent callus stage to regenerate at least one haploid, polyhaploid and/or doubled haploid callus, cell, plant or seed. Thus, a large portion of the plants obtained by the method according to the invention can be directly used for breeding without the need to apply chromosome doubling agents.

In a preferred embodiment of the method described above, a population of plants is regenerated in step iv), which undergoes spontaneous chromosome doubling at a rate of at least 40%, at least 50%, at least 60% or at least 70%. In this context, a population comprises at least 10 individuals.

In a further preferred embodiment of the method described above, before regeneration in step iv) the structure of the callus or the embryo may be destructed by applying a physical force, e.g., the callus or the embryo may be divided into pieces, may be crushed or mashed.

In one embodiment of the method described above, step i) comprises one or more of the following steps:
(a) providing a bud or an inflorescence or a part thereof comprising microspores at the tetrad and uninuclear development stage,
(b) optionally, disinfecting the bud or an inflorescence or the part thereof of (a),
(c) transferring the bud or an inflorescence or the part thereof of (a) or (b) to medium containing macro- and microsalts, saccharides and a complex protein composition,
(d) homogenizing the medium containing the bud or an inflorescence or the part thereof of step (c) and thereby producing a cell suspension,
(e) optionally, purifying the cell suspension preferably by means of a strainer and/or centrifugation thereby
(f) producing a suspension of microspores with a density of around 10,000 to 500,000 microspores per ml medium,
(g) optionally, cultivating the microspore suspension in the dark, and
(h) adding at least one histone deacetylase inhibitor (HDACi) to a final concentration of 1 nM to 10 μM, preferably 100 nM to 10 μM, more preferably 1 μM to 10 μM.

In a preferred embodiment, in step (a) the bud or the inflorescence or the part thereof are removed from a Beta vulgaris plant; inflorescence may be removed before the flower opens. A part of the inflorescence may be the tip of an inflorescence branch. The removed bud or inflorescence or the part thereof may be stored intermediately at room temperature or at chilling temperatures, for example 2 to 10° C. or 4 to 8° C.

In a preferred embodiment, in step (c) the saccharide is a monosaccharide such as glucose monohydrate. In another preferred embodiment, the medium of step (c) may comprise additionally one or more amino acid such as glutamine and/or nucleoside such as uridine and cytidine. Each of these additional components can be present in concentrations between 20 and 750 mg/l.

In a further embodiment of the method described above, step i) comprises all steps (a)-(h).

In a preferred embodiment of the method described above, the cell suspension is purified by means of a strainer in step (e) by subsequently passing it through a cell strainer with a pore size in a range from about 50 to 100 μm and through a cell strainer with a pore size in a range from about 25 to 50 μm. Preferably, passing through each of the strainers with different pore sizes is performed at least 1 to 3 times or exactly 1 to 3 times. Alternatively, the cell suspension may be purified by means of a strainer in step (E) by passing it through a cell strainer with a pore size in a range from about 25 to 100 μm repeatedly, preferably at least 1 to 6 times or exactly 1 to 6 times.

In one embodiment of the method according to any of the embodiments described above, the step ii) of the method further comprises one or more of the following steps:
(i) optionally, cultivating the microspore suspension in the dark, and
(j) adding at least one histone deacetylase inhibitor (HDACi) to a final concentration of 1 nM to 10 μM, preferably 100 nM to 10 μM, more preferably 1 μM to 10 μM, and
(k) removing the at least one histone deacetylase inhibitor (HDACi) from the microspore culture of step ii).

In one embodiment of the method described above, step ii) comprises all steps (i)-(k). Removing the HDACi prior to the culturing to obtain a callus leads to particularly efficient callus formation. Removing of HDACi may be conducted by a step of centrifugation, washing of the microspores treated with HDACi, or replacing of the cultivation medium by the same medium without the HDACi or a different medium without the HDACi, or any combination of these steps. Such steps for removing the HDACi may be applied once, repeated or several times, prior to and/or during callus induction.

In any of the embodiments of the methods described above, the at least one histone deacetylase inhibitor (HDACi) is selected from the group consisting of trichostatin A (TSA), hydroxamic acids and hydroxamates, such as vorinostat (SAHA), belinostat (PXD101), dacinostat (LAQ824), and panobinostat (LBH589), cyclic tetrapeptides, such as trapoxin B and depsipeptides, such as romidepsin (FK228), benzamides such as entinostat (MS-275), tacedinaline (C1994), and mocetinostat (MGCD0103), electrophilic ketones, and aliphatic acid compounds such as phenylbutyrate and valproic acid, preferably the histone deacetylase inhibitor (HDACi) is trichostatin A (TSA) or romidepsin (FK228).

A range of different HDACis are known to the skilled person. Good results are in particular obtained with TSA.

The complex protein composition can be obtained from any protein source such as milk, meat, fish, cereal or other plants. Good results are in particular obtained with a protein composition derived from milk and subjected to at least partial hydrolysis.

In any of the embodiments of the method described above, the complex protein composition comprises or consists of hydrolysed or partially hydrolysed protein matter derived from milk, such as casein or whey, animals, such as meat or fish, cereal, such as rice or corn, plants, such as soybean or combinations thereof.

Preferably, in any of the embodiments of the method described above, the complex protein composition comprises or consists of hydrolysed milk protein isolates, hydrolysed lactoprotein concentrate, hydrolysed casein isolates, casein hydrolysates, hydrolysed lactalbumin, hydrolysed casein sodium, hydrolysed calcium caseinate, hydrolysed full cow's milk, partially or completely skimmed milk, hydrolysed soy protein isolate, hydrolysed soybean concentrate or combinations thereof.

Particularly preferably, in any of the embodiments of the method described above, the complex protein composition comprises or consists of a proteolysate selected from the group consisting of casein hydrolysate, soybean hydrolysate, rice proteolysate, potato protein hydrolysate, fish protein hydrolysate, ovalbumin hydrolysate, lactalbumin hydrolysate, glutin hydrolysate, animal and plant proteolysate and a combination thereof.

Further preferably, in the complex protein composition, the hydrolysis degree is in a range from about 20 to about 80%, preferably from about 30 to about 80%, particularly preferably from about 40 to about 60%.

In one embodiment of the method according to any of the embodiments described above, the at least one histone deacetylase inhibitor (HDACi) is present at a concentration from about 1 nM to about 10 μM, preferably 100 nM to 10 μM, more preferably 1 μM to 10 μM in the culture medium used in step ii).

In another embodiment of the method according to any of the embodiments described above, the complex protein composition is present at a concentration from about 100 to about 20,000 mg/l, preferably about 500 to about 15,000 mg/l, particularly preferably about 2,000 to about 10,000 mg/l in the culture medium used in step ii).

Regenerating a plant in step iv) may involve the use of one or more plant growth regulators.

In one embodiment of the method according to any of the embodiments described above, the callus or embryo is contacted with one or more plant growth regulator(s) selected from auxins, cytokinins, gibberellins, abscisic acid and mixtures thereof in step iv).

In the plants obtained according to the present invention, chromosome doubling can occur either spontaneously or it can be promoted, facilitated or enhanced by a chromosome doubling agent.

In one embodiment of the method according to any of the embodiments described above, one or more chromosome doubling agent(s) is/are added during step iii) and/or step iv). In particular, colchicine may be added during step iii) and/or step iv).

The present invention also relates to a method for preparing microspores of a Beta vulgaris plant for generation of haploid, polyhaploid and/or doubled haploid haploid plants, comprising the steps:
(A) providing a bud or an inflorescence or a part thereof comprising microspores at the tetrad and uninuclear development stage,
(B) optionally, disinfecting the bud or an inflorescence or the part thereof of (a),
(C) transferring the bud or an inflorescence or the part thereof of (a) or (b) to medium containing macro- and microsalts, saccharides and a complex protein composition,
(D) homogenizing the medium containing the bud or an inflorescence or the part thereof of step (c) and thereby producing a cell suspension,
(E) optionally, purifying the cell suspension preferably by means of a strainer and/or centrifugation thereby
(F) producing a suspension of microspores with a density of 10 to 1000 k microspores per ml medium, preferably around 10,000 to 500,000 microspores per ml medium,
(G) optionally, cultivating the microspore suspension in the dark, and
(H) adding at least one histone deacetylase inhibitor (HDACi) to a final concentration of 1 nM to 10 μM, preferably 100 nM to 10 μM, more preferably 1 μM to 10 μM,
(I) optionally, removing the at least one histone deacetylase inhibitor (HDACi) from the microspore culture.

In a preferred embodiment, in step (A) the bud or the inflorescence or the part thereof are removed from a Beta vulgaris plant; inflorescence may be removed before the flower opens. A part of the inflorescence may be the tip of an inflorescence branch. The removed bud or inflorescence or the part thereof may be stored intermediately at room temperature or at chilling temperatures, for example 2 to 10° C. or 4 to 8° C.

In a preferred embodiment, in step (C) the saccharide is a monosaccharide such as glucose monohydrate. In another preferred embodiment, the medium of step (c) may comprise additionally one or more amino acid such as glutamine and/or nucleoside such as uridine and cytidine. Each of these additional components can be present in concentrations between 20 and 750 mg/l.

In a preferred embodiment of the method described above, the cell suspension is purified by means of a strainer in step (E) by subsequently passing it through a cell strainer with a pore size in a range from about 50 to 100 μm and through a cell strainer with a pore size in a range from about 25 to 50 μm. Preferably, passing through each of the strainers with different pore sizes is performed at least 1 to 3 times or exactly 1 to 3 times. Alternatively, the cell suspension may be purified by means of a strainer in step (E) by passing it through a cell strainer with a pore size in a range from about 25 to 100 μm repeatedly, preferably at least 1 to 6 times or exactly 1 to 6 times.

In one embodiment of the method for preparing microspores according to any of the embodiments described above, the at least one histone deacetylase inhibitor (HDACi) is selected from the group consisting of trichostatin A (TSA), hydroxamic acids and hydroxamates, such as vorinostat (SAHA), belinostat (PXD101), dacinostat (LAQ824), and panobinostat (LBH589), cyclic tetrapeptides, such as trapoxin B and depsipeptides, such as romidepsin (FK228), benzamides such as entinostat (MS-275), tacedinaline (C1994), and mocetinostat (MGCD0103), electrophilic ketones, and aliphatic acid compounds such as phenylbutyrate and valproic acid, preferably the histone deacetylase inhibitor (HDACi) is trichostatin A (TSA) or romidepsin (FK228).

In another embodiment of the method for preparing microspores according to any of the embodiments described above, the complex protein composition comprises or consists of hydrolysed or partially hydrolysed protein matter derived from milk, such as casein or whey, animals, such as meat or fish, cereal, such as rice or corn, plants, such as soybean or combinations thereof.

Preferably, the complex protein composition comprises or consists of hydrolysed milk protein isolates, hydrolysed lactoprotein concentrate, hydrolysed casein isolates, casein hydrolysates, hydrolysed lactalbumin, hydrolysed casein sodium, hydrolysed calcium caseinate, hydrolysed full cow's milk, partially or completely skimmed milk, hydrolysed soy protein isolate, hydrolysed soybean concentrate or combinations thereof.

Particularly preferably, the complex protein composition comprises or consists of a proteolysate selected from the group consisting of casein hydrolysate, soybean hydrolysate, rice proteolysate, potato protein hydrolysate, fish protein hydrolysate, ovalbumin hydrolysate, lactalbumin hydrolysate, glutin hydrolysate, animal and plant proteolysate and a combination thereof.

Further preferably, the hydrolysis degree of the complex protein composition is in a range from about 20 to about 80%, preferably from about 30 to about 80%, particularly preferably from about 40 to about 60%.

In one embodiment of the method for preparing microspores according to any of the embodiments described above, the complex protein composition is present at a concentration from about 100 to about 20,000 mg/l, preferably about 500 to about 15,000 mg/l, particularly preferably about 2,000 to about 10,000 mg/l in the culture medium used in step c).

In another aspect, the present invention relates to a kit for producing a haploid, polyhaploid and/or doubled haploid embryo, callus, seed and/or plant of the species Beta vulgaris from at least one isolated microspore comprising:
(a) at least one histone deacetylase inhibitor (HDACi), and
(b) a complex protein composition,

wherein the at least one histone deacetylase inhibitor (HDACi) and the complex protein composition are comprised within the same container or within two or more separate containers.

In one embodiment, the kit comprises a first composition comprising an HDACi and a second composition comprising a complex protein composition. In one embodiment, the first and second compositions are media for plant cell culture. In one embodiment, the first composition is in a first container and the second composition is in a second container. In another embodiment, the kit comprises a composition comprising the HDACi and the complex protein composition. In one embodiment, the composition is a medium for plant cell culture. The kit may include a set of instructions for using the HDACi and/or the complex protein composition. Either one or both of the HDACi and the complex protein composition may be in a concentrated form and require dilution prior to use.

In one embodiment of the kit according to any of the embodiments described above, the at least one histone deacetylase inhibitor (HDACi) is selected from the group consisting of trichostatin A (TSA), hydroxamic acids and hydroxamates, such as vorinostat (SAHA), belinostat (PXD101), dacinostat (LAQ824), and panobinostat (LBH589), cyclic tetrapeptides, such as trapoxin B and depsipeptides, such as romidepsin (FK228), benzamides such as entinostat (MS-275), tacedinaline (C1994), and mocetinostat (MGCD0103), electrophilic ketones, and aliphatic acid compounds such as phenylbutyrate and valproic acid, preferably the histone deacetylase inhibitor (HDACi) is trichostatin A (TSA) or romidepsin (FK228).

In another embodiment of the kit according to any of the embodiments described above, the complex protein composition comprises or consists of hydrolysed or partially hydrolysed protein matter derived from milk, such as casein or whey, animals, such as meat or fish, cereal, such as rice or corn, plants, such as soybean or combinations thereof.

Preferably, the complex protein composition comprises or consists of hydrolysed milk protein isolates, hydrolysed lactoprotein concentrate, hydrolysed casein isolates, casein hydrolysates, hydrolysed lactalbumin, hydrolysed casein sodium, hydrolysed calcium caseinate, hydrolysed full cow's milk, partially or completely skimmed milk, hydrolysed soy protein isolate, hydrolysed soybean concentrate or combinations thereof.

Particularly preferably, the complex protein composition comprises or consists of a proteolysate selected from the group consisting of casein hydrolysate, soybean hydrolysate, rice proteolysate, potato protein hydrolysate, fish protein hydrolysate, ovalbumin hydrolysate, lactalbumin hydrolysate, glutin hydrolysate, animal and plant proteolysate and a combination thereof.

In one embodiment, the hydrolysis degree is in a range from 20 to 80%, preferably 30 to 80%, particularly preferably 40 to 60%.

In another embodiment of the kit according to any of the embodiments described above, the kit further comprises one or more plant growth regulator(s) selected from auxins, cytokinins, gibberellins, abscisic acid, and mixtures thereof.

The one or more plant growth regulators can be comprised in the same container as the HDACi and/or the complex protein composition or in a separate container.

In another embodiment, the kit further comprises one or more chromosome doubling agent(s) such as colchicine.

The present invention also relates to the use of a method or kit according to the present invention for producing haploid, polyhaploid and/or doubled haploid plants of the species Beta vulgaris by androgenesis from isolated microspores.

The present invention also relates to the use of a HDACi and a complex protein composition for producing haploid, polyhaploid and/or doubled haploid embryos and/or plants of the species Beta vulgaris by androgenesis from isolated microspores.

In one embodiment, the present invention relates to the use of a histone deacetylase inhibitor (HDACi) and a complex protein composition or the use of a kit as defined in any of the embodiments described above for producing a haploid, polyhaploid and/or doubled haploid embryo, callus and/or plant or seed of the species Beta vulgaris.

Preferably, the histone deacetylase inhibitor (HDACi) and the complex protein composition or the kit are used in a method according to any of the embodiments described above.

In one embodiment of the use described above, the histone deacetylase inhibitor (HDACi) is selected from the group consisting of trichostatin A (TSA), hydroxamic acids and hydroxamates, such as vorinostat (SAHA), belinostat (PXD101), dacinostat (LAQ824), and panobinostat (LBH589), cyclic tetrapeptides, such as trapoxin B and depsipeptides, such as romidepsin (FK228), benzamides such as entinostat (MS-275), tacedinaline (C1994), and mocetinostat (MGCD0103), electrophilic ketones, and aliphatic acid compounds such as phenylbutyrate and valproic acid, preferably the histone deacetylase inhibitor (HDACi) is trichostatin A (TSA) or romidepsin (FK228).

In another embodiment of the use according to any of the embodiments described above, the complex protein composition comprises or consists of hydrolysed or partially hydrolysed protein matter derived from milk, such as casein or whey, animals, such as meat or fish, cereal, such as rice or corn, plants, such as soybean or combinations thereof.

Preferably, the complex protein composition comprises or consists of hydrolysed milk protein isolates, hydrolysed lactoprotein concentrate, hydrolysed casein isolates, casein hydrolysates, hydrolysed lactalbumin, hydrolysed casein sodium, hydrolysed calcium caseinate, hydrolysed full cow's milk, partially or completely skimmed milk, hydrolysed soy protein isolate, hydrolysed soybean concentrate or combinations thereof.

Particularly preferably, the complex protein composition comprises or consists of a proteolysate selected from the group consisting of casein hydrolysate, soybean hydrolysate, rice proteolysate, potato protein hydrolysate, fish protein hydrolysate, ovalbumin hydrolysate, lactalbumin hydrolysate, glutin hydrolysate, animal and plant proteolysate and a combination thereof.

In a preferred embodiment of the use described above, the hydrolysis degree of the complex protein composition is in a range from 20 to 80%, preferably 30 to 80%, particularly preferably 40 to 60%.

In yet another aspect, the present invention relates to a population of haploid, polyhaploid and/or doubled haploid Beta vulgaris plants directly derived from a single flower, single inflorescence or single bud, preferably obtained or obtainable by a method according to any of the embodiments described above. The haploid, polyhaploid and/or doubled haploid Beta vulgaris plants, or the population thereof, is thus obtained by an artificial step of extracting a microspore and culturing the same ex vivo, i.e., outside of the plant environment, and is treated with at least one HDACi compound. All haploid, polyhaploid and/or doubled haploid Beta vulgaris plants, or a population thereof, obtained by the methods disclosed herein are thus represent cells or plants not exclusively obtained by means of an essentially biological process, as the generation process inherently includes several steps of human intervention not occurring in nature in the form of microspore isolation and a chemical treatment.

Advantageously, the method of the present invention allows to obtain a large number of haploid, polyhaploid and/or doubled haploid Beta vulgaris plants from a single flower, single inflorescence or single bud, while using ovule culturing methods, a maximum of four plants can be obtained from one flower, one inflorescence or one bud.

In one embodiment of the population described above, the population comprises at least 10 individuals, preferably at least 50 individuals, more preferably at least 100 individuals.

A further advantage is that the plants obtained by the method according to the invention show spontaneous chromosome doubling rates of over 50%, while plants obtained by ovule culture only show a maximum of 5% spontaneous doubling. Thus, a large portion of the plants obtained can be directly used for breeding purposes without the need to apply chromosome doubling agents.

In another embodiment of the population according to any of the embodiments described above, the population comprises at least 40%, at least 50%, at least 60% or at least 70% doubled haploid plants.

The present invention also relates to the use of a histone deacetylase inhibitor (HDACi) and a complex protein composition or the use of a kit as defined in any of the embodiments described above for increasing the spontaneous chromosome doubling rate in a population of Beta vulgaris plants derived from one single flower, single inflorescence or single bud and obtained by microspore culture.

In a preferred embodiment of the use described above, the histone deacetylase inhibitor (HDACi) and the complex protein composition or the kit as defined in any of the embodiments described above is used in a method for producing a haploid, polyhaploid and/or doubled haploid embryo, callus, seed and/or plant of the species Beta vulgaris according to any of the embodiments described above.

In one embodiment of the use, the histone deacetylase inhibitor (HDACi) is selected from the group consisting of trichostatin A (TSA), hydroxamic acids and hydroxamates, such as vorinostat (SAHA), belinostat (PXD101), dacinostat (LAQ824), and panobinostat (LBH589), cyclic tetrapeptides, such as trapoxin B and depsipeptides, such as romidepsin (FK228), benzamides such as entinostat (MS-275), tacedinaline (C1994), and mocetinostat (MGCD0103), electrophilic ketones, and aliphatic acid compounds such as phenylbutyrate and valproic acid, preferably the histone deacetylase inhibitor (HDACi) is trichostatin A (TSA) or romidepsin (FK228).

In another embodiment of the use according to any of the embodiments described above, the complex protein composition comprises or consists of hydrolysed or partially hydrolysed protein matter derived from milk, such as casein or whey, animals, such as meat or fish, cereal, such as rice or corn, plants, such as soybean or combinations thereof.

Preferably, the complex protein composition comprises or consists of hydrolysed milk protein isolates, hydrolysed lactoprotein concentrate, hydrolysed casein isolates, casein hydrolysates, hydrolysed lactalbumin, hydrolysed casein sodium, hydrolysed calcium caseinate, hydrolysed full cow's milk, partially or completely skimmed milk, hydrolysed soy protein isolate, hydrolysed soybean concentrate or combinations thereof.

Particularly preferably, the complex protein composition comprises or consists of a proteolysate selected from the group consisting of casein hydrolysate, soybean hydrolysate, rice proteolysate, potato protein hydrolysate, fish protein hydrolysate, ovalbumin hydrolysate, lactalbumin hydrolysate, glutin hydrolysate, animal and plant proteolysate and a combination thereof.

In a preferred embodiment of the use described above, the hydrolysis degree of the complex protein composition is in a range from 20 to 80%, preferably 30 to 80%, particularly preferably 40 to 60%.

The present invention also relates to a method for increasing the rate of spontaneous chromosome doubling in a population of Beta vulgaris plants obtained from one single flower, single inflorescence or single bud comprising:
i) providing at least one isolated microspore from at least one single flower, single inflorescence or single bud of Beta vulgaris,
ii) culturing the at least one microspore from step i) in the presence of a complex protein composition and at least one histone deacetylase inhibitor (HDACi),
iii) culturing the at least one microspore from step ii) to obtain a callus or an embryo, and
iv) regenerating and thereby obtaining a population of plants from the callus or embryo of step iii).

Preferably, in step i) microspores from at least one single flower, single inflorescence or single bud of Beta vulgaris Have been provided in a density of at least 10,000 microspores per ml, more preferred a density of at least 25,000 microspores per ml or at least 50,000 microspores per ml, even more preferred a density of at least 100,000 microspores per ml, most preferred at least 200,000 microspores per ml.

In a further preferred embodiment of the method described above, before regeneration in step iv) the structure of the callus or the embryo may be destructed by applying a physical force, e.g., the callus or the embryo may be divided into pieces, may be crushed or mashed.

Preferably, the population of plants regenerated in step iv) comprises at least 10 individuals, at least 50 individuals or at least 100 individuals.

In a preferred embodiment of the method described above, the population regenerated in step iv) undergoes spontaneous chromosome doubling at a rate of at least 40%, at least 50%, at least 60% or at least 70%.

In one embodiment of the method for increasing the rate of spontaneous chromosome doubling according to any of the embodiments described above, step i) comprises the steps:
(a) providing a bud or an inflorescence or a part thereof comprising microspores at the tetrad and uninuclear development stage,
(b) optionally, disinfecting the bud or an inflorescence or the part thereof of (a),
(c) transferring the bud or an inflorescence or the part thereof of (a) or (b) to medium containing macro- and microsalts, saccharides and a complex protein composition,
(d) homogenizing the medium containing the bud or an inflorescence or the part thereof of step (c) and thereby producing a cell suspension,
(e) optionally, purifying the cell suspension preferably by means of a strainer and/or centrifugation thereby
(f) producing a suspension of microspores with a density of around 10,000 to 500,000 microspores per ml medium,
(g) optionally, cultivating the microspore suspension in the dark, and
(h) adding at least one histone deacetylase inhibitor (HDACi) to a final concentration of 1 nM to 10 μM, preferably 100 nM to 10 μM, more preferably 1 μM to 10 μM.

In a preferred embodiment, in step (a) the bud or the inflorescence or the part thereof are removed from a Beta vulgaris plant; inflorescence may be removed before the flower opens. A part of the inflorescence may be the tip of an inflorescence branch. The removed bud or inflorescence or the part thereof may be stored intermediately at room temperature or at chilling temperatures, for example 2 to 10° C. or 4 to 8° C.

In a preferred embodiment, in step (c) the saccharide is a monosaccharide such as glucose monohydrate. In another preferred embodiment, the medium of step (c) may comprise additionally one or more amino acid such as glutamine and/or nucleoside such as uridine and cytidine. Each of these additional components can be present in concentrations between 20 and 750 mg/l.

In another embodiment of the method for increasing the rate of spontaneous chromosome doubling according to any of the embodiments described above, the at least one histone deacetylase inhibitor (HDACi) is selected from the group consisting of trichostatin A (TSA), hydroxamic acids and hydroxamates, such as vorinostat (SAHA), belinostat (PXD101), dacinostat (LAQ824), and panobinostat (LBH589), cyclic tetrapeptides, such as trapoxin B and depsipeptides, such as romidepsin (FK228), benzamides such as entinostat (MS-275), tacedinaline (C1994), and mocetinostat (MGCD0103), electrophilic ketones, and aliphatic acid compounds such as phenylbutyrate and valproic acid, preferably the histone deacetylase inhibitor (HDACi) is trichostatin A (TSA) or romidepsin (FK228). In a further embodiment of the method for increasing the rate of spontaneous chromosome doubling according to any of the embodiments described above, the complex protein composition comprises or consists of hydrolysed or partially hydrolysed protein matter derived from milk, such as casein or whey, animals, such as meat or fish, cereal, such as rice or corn, plants, such as soybean or combinations thereof.

Preferably, the complex protein composition comprises or consists of hydrolysed milk protein isolates, hydrolysed lactoprotein concentrate, hydrolysed casein isolates, casein hydrolysates, hydrolysed lactalbumin, hydrolysed casein sodium, hydrolysed calcium caseinate, hydrolysed full cow's milk, partially or completely skimmed milk, hydrolysed soy protein isolate, hydrolysed soybean concentrate or combinations thereof.

Particularly preferably, the complex protein composition comprises or consists of a proteolysate selected from the group consisting of casein hydrolysate, soybean hydrolysate, rice proteolysate, potato protein hydrolysate, fish protein hydrolysate, ovalbumin hydrolysate, lactalbumin hydrolysate, glutin hydrolysate, animal and plant proteolysate and a combination thereof.

In one embodiment, the hydrolysis degree of the complex protein composition is in a range from about 20 to about 80%, preferably from about 30 to about 80%, particularly preferably from about 40 to about 60%.

In another embodiment of the method for increasing the rate of spontaneous chromosome doubling according to any of the embodiments described above, the at least one histone deacetylase inhibitor (HDACi) is present at a concentration from about 1 nM to about 10 μM, preferably 100 nM to 10 μM, more preferably 1 μM to 10 μM in the culture medium used in step ii).

In a further embodiment of the method for increasing the rate of spontaneous chromosome doubling according to any of the embodiments described above, the complex protein composition is present at a concentration from about 100 to about 20,000 mg/l, preferably about 500 to about 15,000 mg/l, particularly preferably about 2,000 to about 10,000 mg/l in the culture medium used in step ii).

In one embodiment of the method for increasing the rate of spontaneous chromosome doubling according to any of the embodiments described above, the callus or embryo is contacted with one or more plant growth regulator(s) selected from auxins, cytokinins, gibberellins, abscisic acid and mixtures thereof in step iv).

The doubled haploid plants obtained by the methods described above are fertile and can be directly used for further breeding with other plants as maternal or paternal crossing partner, preferably for the development of a OH line/DH population or for hybrid crossing.

The present invention therefore also provides a method for producing a hybrid Beta vulgaris plant comprising the crossing of a doubled haploid plant obtained or obtainable by microspore culture with another plant of the same species with a different genotype, and optionally obtaining, identifying or selecting seeds grown on the maternal parent of the cross or obtaining, identifying or selecting progeny plants from said cross.

In one aspect, the present invention relates to a method for producing a OH population or OH line of Beta vulgaris plants comprising the steps:
a) providing a doubled haploid Beta vulgaris plant by microspore culture,
b) propagate the doubled haploid plant from step a) by selfing, and
c) obtaining a progeny seed or plant.

In one embodiment of the method for producing a OH population or OH line of Beta vulgaris plants described above, step a) comprises the steps:
i) providing at least one isolated microspore of Beta vulgaris,
ii) culturing the at least one microspore from step i) in the presence of a complex protein composition and at least one histone deacetylase inhibitor (HDACi),
iii) culturing the at least one microspore from step ii) to obtain a callus or an embryo, and
iv) regenerating and thereby obtaining at least one plant from the callus or embryo of step iii).

In a further preferred embodiment of the method described above, before regeneration in step iv) the structure of the callus or the embryo may be destructed by applying a physical force, e.g., the callus or the embryo may be divided into pieces, may be crushed or mashed.

In one embodiment of the method described above, step i) comprises one or more of the following steps:
(a) providing a bud or an inflorescence or a part thereof comprising microspores at the tetrad and uninuclear development stage,
(b) optionally, disinfecting the bud or an inflorescence or the part thereof of (a),
(c) transferring the bud or an inflorescence or the part thereof of (a) or (b) to medium containing macro- and microsalts, saccharides and a complex protein composition,
(d) homogenizing the medium containing the bud or an inflorescence or the part thereof of step (c) and thereby producing a cell suspension,
(e) optionally, purifying the cell suspension preferably by means of a strainer and/or centrifugation thereby
(f) producing a suspension of microspores with a density of around 10,000 to 500,000 microspores per ml medium,
(g) optionally, cultivating the microspore suspension in the dark, and
(h) adding at least one histone deacetylase inhibitor (HDACi) to a final concentration of 1 nM to 10 μM, preferably 100 nM to 10 μM, more preferably 1 μM to 10 μM.

In a preferred embodiment, in step (a) the bud or the inflorescence or the part thereof are removed from a Beta vulgaris plant; inflorescence may be removed before the flower opens. A part of the inflorescence may be the tip of an inflorescence branch. The removed bud or inflorescence or the part thereof may be stored intermediately at room temperature or at chilling temperatures, for example 2 to 10° C. or 4 to 8° C.

In a preferred embodiment, in step (c) the saccharide is a monosaccharide such as glucose monohydrate. In another preferred embodiment, the medium of step (c) may comprise additionally one or more amino acid such as glutamine and/or nucleoside such as uridine and cytidine. Each of these additional components can be present in concentrations between 20 and 750 mg/l.

In a further embodiment of the method described above, step i) comprises all steps (a)-(h).

In another embodiment of the method for producing a OH population or OH line of Beta vulgaris plants according to any of the embodiments described above, the step ii) of the method further comprises one or more of the following steps:
(i) optionally, cultivating the microspore suspension in the dark,
(j) adding at least one histone deacetylase inhibitor (HDACi) to a final concentration of 1 nM to 10 μM, preferably 100 nM to 10 μM, more preferably 1 μM to 10 μM and
(k) removing the at least one histone deacetylase inhibitor (HDACi) from the microspore culture of step ii).

In a further embodiment of the method described above, step ii) comprises all steps (i)-(k).

In one embodiment of the method for producing a OH population or OH line of Beta vulgaris plants according to any of the embodiments described above, the at least one histone deacetylase inhibitor (HDACi) is selected from the group consisting of trichostatin A (TSA), hydroxamic acids and hydroxamates, such as vorinostat (SAHA), belinostat (PXD101), dacinostat (LAQ824), and panobinostat (LBH589), cyclic tetrapeptides, such as trapoxin B and depsipeptides, such as romidepsin (FK228), benzamides such as entinostat (MS-275), tacedinaline (C1994), and mocetinostat (MGCD0103), electrophilic ketones, and aliphatic acid compounds such as phenylbutyrate and valproic acid, preferably the histone deacetylase inhibitor (HDACi) is trichostatin A (TSA) or romidepsin (FK228).

In another embodiment of the method for producing a OH population or OH line of Beta vulgaris plants according to any of the embodiments described above, the complex protein composition comprises or consists of hydrolysed or partially hydrolysed protein matter derived from milk, such as casein or whey, animals, such as meat or fish, cereal, such as rice or corn, plants, such as soybean or combinations thereof.

Preferably, the complex protein composition comprises or consists of hydrolysed milk protein isolates, hydrolysed lactoprotein concentrate, hydrolysed casein isolates, casein hydrolysates, hydrolysed lactalbumin, hydrolysed casein sodium, hydrolysed calcium caseinate, hydrolysed full cow's milk, partially or completely skimmed milk, hydrolysed soy protein isolate, hydrolysed soybean concentrate or combinations thereof.

Particularly preferably, the complex protein composition comprises or consists of a proteolysate selected from the group consisting of casein hydrolysate, soybean hydrolysate, rice proteolysate, potato protein hydrolysate, fish protein hydrolysate, ovalbumin hydrolysate, lactalbumin hydrolysate, glutin hydrolysate, animal and plant proteolysate and a combination thereof.

In one embodiment, the hydrolysis degree of the complex protein mixture is in a range from about 20 to about 80%, preferably from about 30 to about 80%, particularly preferably from about 40 to about 60%.

In another embodiment of the method for producing a OH population or OH line of Beta vulgaris plants according to any of the embodiments described above, the at least one histone deacetylase inhibitor (HDACi) is present at a concentration from about 1 nM to about 10 μM, preferably 100 nM to 10 μM, more preferably 1 μM to 10 μM in the culture medium used in step ii).

In a further embodiment of the method for producing a OH population or OH line of Beta vulgaris plants according to any of the embodiments described above, the complex protein composition is present at a concentration from about 100 to about 20,000 mg/l, preferably about 500 to about 15,000 mg/l, particularly preferably about 2,000 to about 10,000 mg/l in the culture medium used in step ii).

In one embodiment of the method for producing a OH population or OH line of Beta vulgaris plants according to any of the embodiments described above, the callus or embryo is contacted with one or more plant growth regulator(s) selected from auxins, cytokinins, gibberellins, abscisic acid and mixtures thereof in step iv).

In another embodiment of the method for producing a OH population or OH line of Beta vulgaris plants according to any of the embodiments described above, one or more chromosome doubling agent(s), such as colchicine, is/are added during step iii) and/or step iv).

In one aspect, the present invention relates to a method for producing a hybrid Beta vulgaris plant comprising the steps:
a) providing a doubled haploid Beta vulgaris plant by microspore culture,
b) crossing the doubled haploid plant from step a) with another plant of the same species with a different genotype, and
c) obtaining a hybrid progeny seed or plant.

In one embodiment of the method for producing a hybrid Beta vulgaris plant described above, step a) comprises the steps:
i) providing at least one isolated microspore of Beta vulgaris,
ii) culturing the at least one microspore from step i) in the presence of a complex protein composition and at least one histone deacetylase inhibitor (HDACi),
iii) culturing the at least one microspore from step ii) to obtain a callus or an embryo, and
iv) regenerating and thereby obtaining at least one plant from the callus or embryo of step iii).

In one embodiment of the method described above, step i) comprises one or more of the following steps:
(a) providing a bud or an inflorescence or a part thereof comprising microspores at the tetrad and uninuclear development stage,
(b) optionally, disinfecting the bud or an inflorescence or the part thereof of (a),
(c) transferring the bud or an inflorescence or the part thereof of (a) or (b) to medium containing macro- and microsalts, saccharides and a complex protein composition,
(d) homogenizing the medium containing the bud or an inflorescence or the part thereof of step (c) and thereby producing a cell suspension,
(e) optionally, purifying the cell suspension preferably by means of a strainer and/or centrifugation thereby
(f) producing a suspension of microspores with a density of around 10,000 to 500,000 microspores per ml medium,
(g) optionally, cultivating the microspore suspension in the dark, and
(h) adding at least one histone deacetylase inhibitor (HDACi) to a final concentration of 1 nM to 10 μM, preferably 100 nM to 10 μM, more preferably 1 μM to 10 μM.

In a preferred embodiment, in step (a) the bud or the inflorescence or the part thereof are removed from a Beta vulgaris plant; inflorescence may be removed before the flower opens. A part of the inflorescence may be the tip of an inflorescence branch. The removed bud or inflorescence or the part thereof may be stored intermediately at room temperature or at chilling temperatures, for example 2 to 10° C. or 4 to 8° C.

In a preferred embodiment, in step (c) the saccharide is a monosaccharide such as glucose monohydrate. In another preferred embodiment, the medium of step (c) may comprise additionally one or more amino acid such as glutamine and/or nucleoside such as uridine and cytidine. Each of these additional components can be present in concentrations between 20 and 750 mg/l.

In a further embodiment of the method described above, step i) comprises all steps (a)-(h).

In another embodiment of the method for producing a hybrid Beta vulgaris plant according to any of the embodiments described above, the step ii) of the method further comprises one or more of the following steps:
(l) optionally, cultivating the microspore suspension in the dark,
(m) adding at least one histone deacetylase inhibitor (HDACi) to a final concentration of 1 nM to 10 μM, preferably 100 nM to 10 μM, more preferably 1 μM to 10 μM and
(n) removing the at least one histone deacetylase inhibitor (HDACi) from the microspore culture of step ii).

In a further embodiment of the method described above, step ii) comprises all steps (i)-(k). In one embodiment of the method for producing a hybrid Beta vulgaris plant according to any of the embodiments described above, the at least one histone deacetylase inhibitor (HDACi) is selected from the group consisting of trichostatin A (TSA), hydroxamic acids and hydroxamates, such as vorinostat (SAHA), belinostat (PXD101), dacinostat (LAQ824), and panobinostat (LBH589), cyclic tetrapeptides, such as trapoxin B and depsipeptides, such as romidepsin (FK228), benzamides such as entinostat (MS-275), tacedinaline (C1994), and mocetinostat (MGCD0103), electrophilic ketones, and aliphatic acid compounds such as phenylbutyrate and valproic acid, preferably the histone deacetylase inhibitor (HDACi) is trichostatin A (TSA) or romidepsin (FK228).

In another embodiment of the method for producing a hybrid Beta vulgaris plant according to any of the embodiments described above, the complex protein composition comprises or consists of hydrolysed or partially hydrolysed protein matter derived from milk, such as casein or whey, animals, such as meat or fish, cereal, such as rice or corn, plants, such as soybean or combinations thereof.

Preferably, the complex protein composition comprises or consists of hydrolysed milk protein isolates, hydrolysed lactoprotein concentrate, hydrolysed casein isolates, casein hydrolysates, hydrolysed lactalbumin, hydrolysed casein sodium, hydrolysed calcium caseinate, hydrolysed full cow's milk, partially or completely skimmed milk, hydrolysed soy protein isolate, hydrolysed soybean concentrate or combinations thereof.

Particularly preferably, the complex protein composition comprises or consists of a proteolysate selected from the group consisting of casein hydrolysate, soybean hydrolysate, rice proteolysate, potato protein hydrolysate, fish protein hydrolysate, ovalbumin hydrolysate, lactalbumin hydrolysate, glutin hydrolysate, animal and plant proteolysate and a combination thereof.

In one embodiment, the hydrolysis degree of the complex protein mixture is in a range from about 20 to about 80%, preferably from about 30 to about 80%, particularly preferably from about 40 to about 60%.

In another embodiment of the method for producing a hybrid Beta vulgaris plant according to any of the embodiments described above, the at least one histone deacetylase inhibitor (HDACi) is present at a concentration from about 1 nM to about 10 μM, preferably 100 nM to 10 μM, more preferably 1 μM to 10 μM in the culture medium used in step ii).

In a further embodiment of the method for producing a hybrid Beta vulgaris plant according to any of the embodiments described above, the complex protein composition is present at a concentration from about 100 to about 20,000 mg/l, preferably about 500 to about 15,000 mg/l, particularly preferably about 2,000 to about 10,000 mg/l in the culture medium used in step ii).

In one embodiment of the method for producing a hybrid Beta vulgaris plant according to any of the embodiments described above, the callus or embryo is contacted with one or more plant growth regulator(s) selected from auxins, cytokinins, gibberellins, abscisic acid and mixtures thereof in step iv).

In another embodiment of the method for producing a hybrid Beta vulgaris plant according to any of the embodiments described above, one or more chromosome doubling agent(s), such as colchicine, is/are added during step iii) and/or step iv).

### EXAMPLES

The present invention is also described and demonstrated by way of the following examples. However, the use of these and other examples anywhere in the specification is illustrative only and in no way limits the scope and meaning of the invention or of any exemplified term. Likewise, the invention is not limited to any particular preferred embodiments described here. Indeed, many modifications and variations of the invention may be apparent to those skilled in the art upon reading this specification, and such variations can be made without departing from the invention in spirit or in scope. The invention is therefore to be limited only by the terms of the appended claims along with the full scope of equivalents to which those claims are entitled.

### Example 1

### Donor Plant Growing

Vernalized Beta vulgaris plants, preferably sugar beet, are cultivated under controlled greenhouse conditions at 16° C.-20° C. and 18 h artificial lighting at 75-150 μmol/m²/s minimum. Preferably, insolation is reduced by shading cloth at >900 μmol/m²/s.

Plants are developing inflorescences after about 40 days. Preferably 4-6 cm tips of the inflorescence branches are removed from the plant before the flowers open. Material is stored in the dark until further processing.

### Isolation of Microspores and Preparation of Microspore Solution

Bracteoles are cut off the inflorescence and buds bigger than 1.8 mm are removed. Remaining part is divided into smaller pieces, preferably pieces of 1-1.5 cm. Buds smaller than 1.8 mm contain typically microspores at the tetrad and uninuclear developmental stage, which are the optimal developmental stages for further progressing. However, it should be noted that due to broad variation of different genotypes and donor sources (e.g., monogerm and multigerm), the correlation between the maximum flower bud size and nuclear stages of the microspores needs to be established for each genotype individually. The determination of the nuclear stages is carried out using a fluorescence-based staining method. The method contains fixation of the cells for instances with Ethanol acetic acid and DAPI staining (Kim, Moon-Za, and In-Chang Jang. “Rapid Assessment of Microspore Development Stage in Pepper Using DAPI and Ferric chloride.” Journal of Plant Biotechnology 2.3 (2000): 129-134.).

Surface disinfection takes place preferably by immersing the plant material in 70% ethanol, followed by treatment in NaOCl 3% active Chlorine. Bleach may be removed by with filter sterilized water.

The flower shoot segments are transferred to to isolation buffer, e.g., in a blending device and 40 ml (isolation buffer, Table 1) is added. The Isolation buffer contains macro- and microsalts, amino acids, monosaccharides and one or more complex protein compositions, pH is adjusted to pH 7.5. The complex protein composition or its source comprises preferably hydrolysed or partially hydrolysed protein matter or protein source. It can derive from any source that is known or that be applicable in addition, such as milk (such as, casein, whey), animal (such as, meat, fish), cereal (such as, rice, corn), plant (such as, soybean) or its combination. This proteinoid comprises milk protein isolates, as described herein lactoprotein concentrate, casein isolates, casein hydrolysate, lactalbumin, casein-sodium or calcium caseinate, full cow's milk, partially or completely skimmed milk, soy protein isolate, soybean protein concentrate etc. In one embodiment, complex protein composition comprises the protein source of the milk protein deriving from bovine milk.

In one embodiment, the protein source is a proteolysate composition. It may comprise height protein hydrolysate, and wherein hydrolysis degree is generally at least about 20% most, comprises about 20% to about 80%, and also comprise about 30% to about 80%, be even more preferably about 40% to about 60%. This hydrolysis degree is the degree of hydrolyzed peptide bonds. The proteolysate composition is commercially obtainable, and preferably selected from the group consisting of casein hydrolysate, soybean hydrolysate, rice protolysate, potato protein hydrolysate, fish protein hydrolyzate, ovalbumin hydrolysate, lactalbumin hydrolysate, glutin hydrolysate, animal and plant protolysate, and/or a combination thereof. The concentration of the proteolysate composition in the isolation buffer may range from 100 to 20,000 mg/l, preferably 500 to 15,000 mg/l, more preferably 2,000 to 10,000 mg/l. The concentration may vary according to the hydrolysis degree and the protein content.

TABLE 1
Isolation buffer

| | mg/l |
|---|---|
| Ca(NO₃)₂ × 4 H₂O | 236 |
| KH₂PO₄ | 136 |
| K₂HPO₄ | 174 |
| KNO₃ | 1010 |
| MgSO₄ × 7 H₂O | 247 |
| H₃BO₃ | 10 |
| Glucose monohydrate (Dextrose) | 90000 |
| Complex protein composition such as | 100 to 20000 |
| casein hydrolysate | |

The material is homogenized, e.g., by means of a homogenizer such as a Waring blender or sonication device at around 3000 rpm for 1 min. The crude cell suspension is passed stepwise through cell strainers of pore sizes between 25 μm and 100 μm. The purified suspension is transferred into tubes for subsequent centrifugation at ˜200 g for 3 min.

The supernatant is removed and the pellet is resuspended in a suitable amount of isolation buffer. An aliquot of the cell solution is taken for evaluation of the number of cells per ml by means of a counting chamber. Cell density is adjusted preferably to a cell density of 25,000 to 500,000 cells with TM media.

### Example 2

### Callus Induction

The microspore solution is kept in tubes preferably for 2 days at 26° C., in the dark until the histone deacetylase inhibitors (HDACi) is added. If the HDACi trichostatin A (TSA) is used, it is dissolved in dimethylsulfoxide (DMSO) to a final concentration of between 1 nM and 10 μM, preferably 100 nM to 10 μM, more preferably 1 μM to 10 μM, depending on the genotype and the donor sources (e.g., monogerm and multigerm). Other non-limiting examples of HDACi which may be used belong to the class of hydroxamic acids and hydroxamates, such as vorinostat (SAHA), belinostat (PXD101), LAQ824, and panobinostat (LBH589). Other non-limiting examples of HDACis for use according to the invention include cyclic tetrapeptides (such as trapoxin B) and depsipeptides, such as romidepsin (FK228), benzamides such as entinostat (MS-275), C1994, and mocetinostat (MGCD0103), electrophilic ketones, and aliphatic acid compounds such as phenylbutyrate and valproic acid.

After 1 to 10 days incubation at 32° C. the TSA is removed by centrifugation preferably at ˜200 g. The media is replaced by half strength NLN media with 130 g/I Saccharose (induction media, Table 2).

TABLE 2
Induction media

| | mg/l |
|---|---|
| Ca(NO₃)₂ × 4 H₂O | 250 |
| KH₂PO₄ | 62.5 |
| KNO₃ | 62.5 |
| MgSO₄ | 30.5 |
| H₃BO₃ | 5 |
| ZnSO₄ × 7 H₂O | 5 |
| MnSO₄ × H₂O | 9.48 |
| CuSO₄ × 5 H₂O | 0.0125 |
| CoCl₂ × 6 H₂O | 0.0125 |
| Na₂MoO₄ × 2 H₂O | 0.125 |
| FeNaEDTA | 18.35 |
| myo-Inositol | 100 |
| Glycine | 2 |
| Nicotinsäure (niacin) | 5 |
| Pyridoxine HCL | 0.5 |
| Thiamine HCL | 0.5 |
| Biotin | 0.05 |
| Folic acid | 5 |
| Glutamine | 800 |
| Gluthatione (reduced) | 30 |
| Serine | 100 |
| Saccharose | 130000 |
| Indole-3-Acetic Acid | 0.3 |
| Kinetin | 0.7 |

The microspore solution is transferred into e.g., multiwell plates, with a volume of 500 μl per each well. The plate may be sealed with Nescofilm for further cultivation at 22 to 28° C. in the dark.

Cell division is induced after about 10 days and callus is visible to the naked eye after about 21 days for most genotypes.

### Example 3

### Plant Regeneration

The callus is removed from the liquid media and transferred to a solid MS media with 1 mg/l GA3 and 1 mg/l TDZ) (regeneration media, Table 3) The callus is grown in dark at 26° C. until first proliferation and cell division can be observed.

TABLE 3
Regeneration media

| | mg/l |
|---|---|
| CaCl₂ | 332.2 |
| KH₂PO₄ | 170 |
| KNO₃ | 1900 |
| MgSO₄ | 180.7 |
| NH₄NO₃ | 1650 |
| H₃BO₃ | 6.2 |
| KJ | 0.83 |
| ZnSO₄ × 7 H₂O | 8.6 |
| MnSO₄ × H₂O | 16.9 |
| CuSO₄ × 5 H₂O | 0.025 |
| CoCL₂ × 6 H₂O | 0.025 |
| Na₂MoO₄ × 2 H₂O | 0.25 |
| FeNaEDTA | 36.7 |
| myo-Inositol | 100 |
| Glycine | 2 |
| Nicotinsäure (niacin) | 0.5 |
| Pyridoxine HCL | 0.5 |
| Thiamine HCL | 0.1 |
| Saccharose | 30000 |
| TDZ | 1 |
| GA3 | 1 |

The shoot development is induced after 3-4 weeks (FIG. 3).

Shoots are transferred to a plant development media (Gürel, Songül, E. K. R. E M. Gürel, and Z. E. K. i. Kaya. “Doubled haploid plant production from unpollinated ovules of sugar beet (Beta vulgaris L.).” Plant Cell Reports 19.12 (2000): 1155-1159.).

Advantageously, the method for the production of haploid, polyhaploid and/or doubled haploid Beta vulgaris plants by isolated microspore cultures of the present invention leads to a significant rate of spontaneous chromosome doubling. In one embodiment the rate of spontaneous chromosome doubling is at least 40%, preferably at least 50% and more preferably at least 60%. In a further embodiment the rate of chromosome doubling is further increased by addition of one or more chromosome doubling agent, such as colchicine, during callus induction to the induction media and/or shoot or root regeneration to the development media.

### Example 4

As shown in FIG. 4, where SAHA was used in comparison to TSA (1 μM and 5 μM; in the same buffer as TSA, cf. Examples above, SAHA functions as HDACi, yet not that good as TSA. TSA showed a strong callus induction in all six genotypes as tested (columns from left to right in FIG. 4 corresponding to one genotype each). As can be seen from FIG. 4, SAHA has a good effect on some genotypes (without SAHA no callus induction was observed, data not shown). SAHA is thus also a good candidate, but less effective than TSA and at the same time more toxic. Therefore, in the direct comparison, TSA is the better candidate to proceed with.

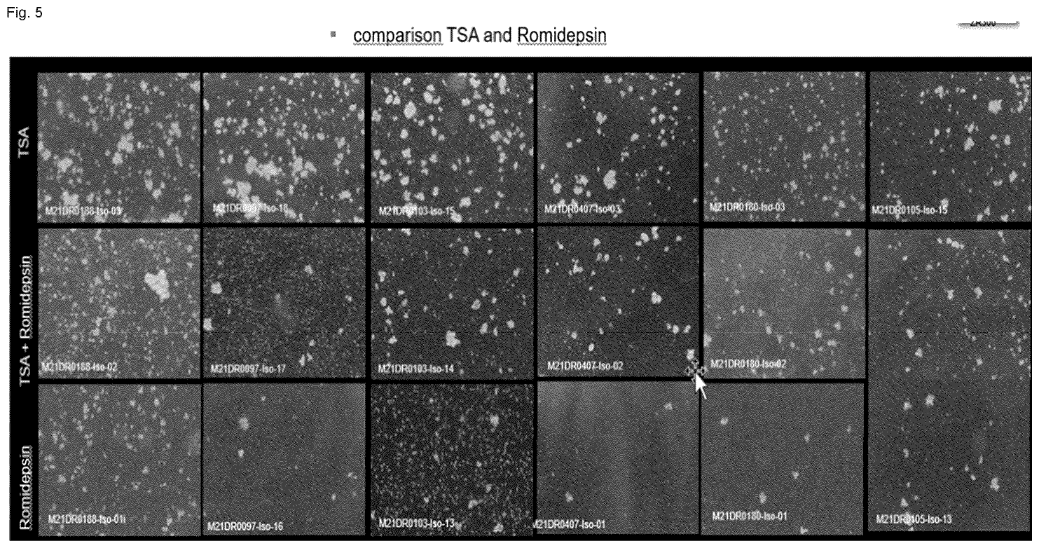

In FIG. 5, romidepsin (2.5 μM) alone or in combination with TSA was tested. Romidepsin alone (bottom row) shows indeed a slight positive effect. Without romidepsin, no callus induction could be observed, as DR (=monogerm) seems to need any additive to generate callus at all. Again, the effect of TSA was superior to that of romidepsin. Further, no clear synergistic effect was observed.

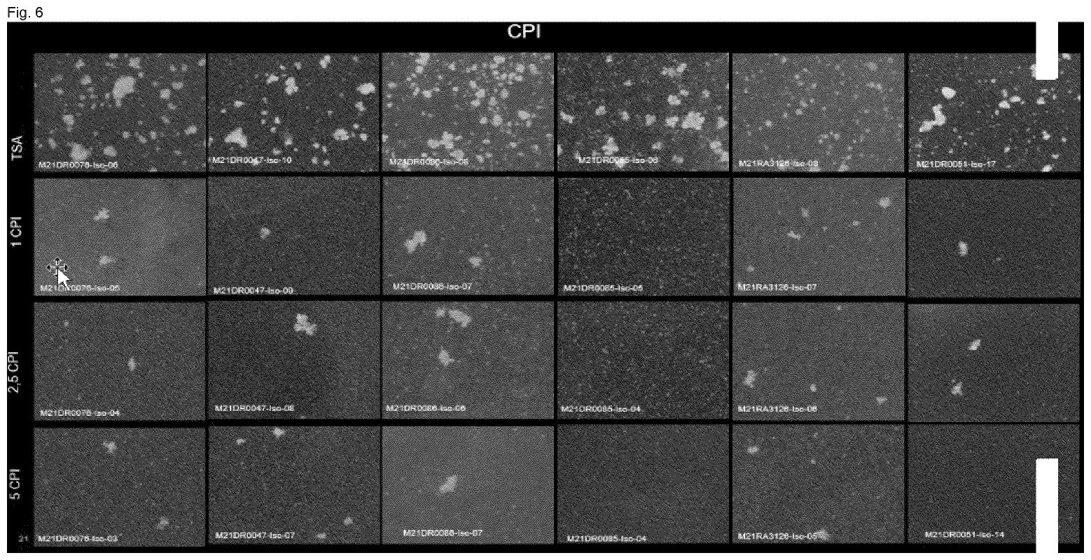

In FIG. 6, CPI-203 results tested on 6 genotypes in the concentration of 1 μM, 2.5 μM and 5 μM. CPI indeed had a slight positive effect, but this bromodomain inhibitor performed worse than TSA.

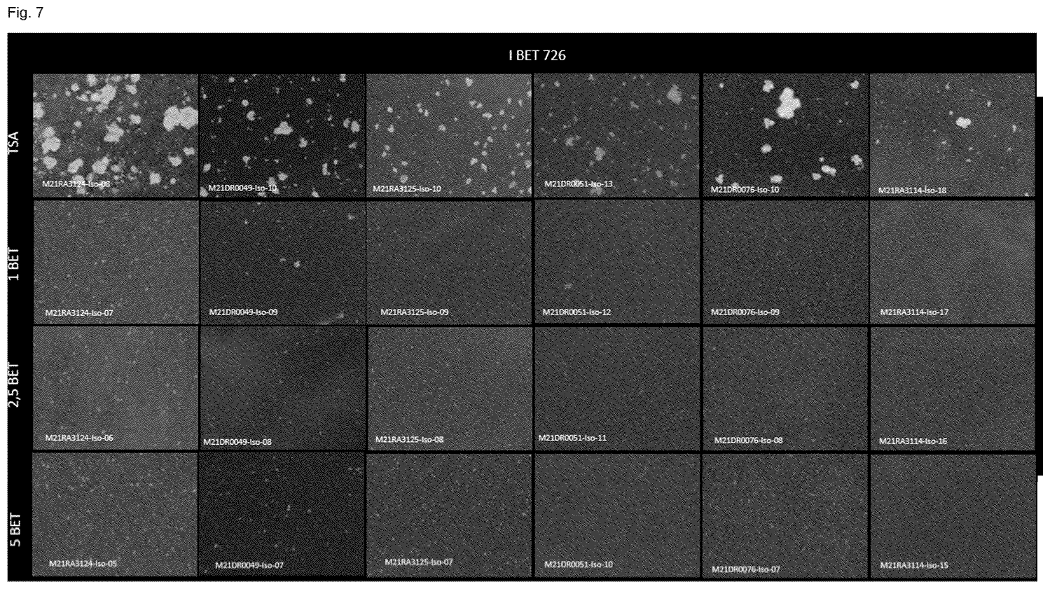

In FIG. 7, the results of testing with the inhibitor I-Bet 726 are documented. Concentrations of 1 μM, 2.5 μM and 5 μM were applied. Notably, I-Bet had a clear negative effect. No callus induction was observed. Again, TSA served as positive control.

In the present example, a TSA concentration of 5 μM was used in all Examples underlying FIG. 4 to FIG. 7 to guarantee a good comparability with the other compounds tested. Notably, TSA generally performed well in the lower μM or even nM range as well, as well as in higher concentrations, as indicated above (data not shown), yet a reasonable mean concentration was used for comparative purposes in this example.

In sum, the HDACi inhibitor TSA performed best in comparison to the inhibitors tested in this Example. Particularly, the HDACi inhibitor performed better in comparison to the bromodoamin inhibitors I-Bet and CPI. In view of the expected effect of HDACi (as well as I.Bet and CPI) on LEC-1 expression, TSA thus seems to be the best candidate. Further, romidepsin and other inhibitors presently under testing have a great potential. Particularly, certain synergistic effects can be expected when combining certain inhibitors.

### LIST OF EMBODIMENTS

The following is a non-exhaustive list of further embodiments encompassed by the present invention.
1. A method for the production of haploid, polyhaploid and/or doubled haploid Beta vulgaris plants by isolated microspore cultures, as disclosed herein.
2. A method for the production of Beta vulgaris calli, embryos, plantlets and plants regenerated from the embryo, progeny of such plants and seed from such plants, usable in or obtained by or obtainable by a method for the production of haploid, polyhaploid and/or doubled haploid Beta vulgaris plants by isolated microspore cultures, as disclosed herein.
3. A method for producing haploid, polyhaploid and/or doubled haploid embryos and/or plants of the species Beta vulgaris from isolated microspores comprising:
a) culturing isolated microspores to obtain a callus or embryo competent for plant regeneration, wherein the microspores have been isolated from plant material of a donor plant of the species Beta vulgaris; and
b) optionally regenerating a plant from the callus or the embryo;
preferably wherein step (a) comprises contacting the microspores with an inhibitor of histone deacetylase (HDACi) and a complex protein composition.
4. The method of any of items 1-3 comprising contacting the microspores with an inhibitor of histone deacetylase (HDACi) and a complex protein composition.
5. The method of any of items 1-4 further comprising contacting the microspores with one or more plant growth regulators selected from auxins, cytokinins, gibberellins, abscisic acid, and mixtures thereof.
6. The method of any of items 1-5 further comprising contacting the microspores with one or more chromosome doubling agent, such as colchicine.
7. A kit for performing a method for producing haploid, polyploid and/or doubled haploid embryos and/or plants of the species Beta vulgaris from isolated microspores, said kit comprising:
an inhibitor of histone deacetylase (HDACi); and
a complex protein composition,
wherein the inhibitor of histone deacetylase (HDACi) and the complex protein composition are comprised within a same container or within two or more separate containers.
8. The kit of item 7, wherein the kit comprises a first composition comprising an HDACi and a second composition comprising a complex protein composition.
9. The kit of item 7 or 8, wherein one or both of the first and second compositions are media for plant cell culture.
10. The kit of any of items 7-9, wherein the first composition is in a first container and the second composition is in a second container.
11. The kit of item 7, wherein the kit comprises a single composition comprising the HDACi and the complex protein composition.
12. The kit of item 11, wherein the single composition is a medium for plant cell culture.
13. The kit of any of items 7-12 further comprising a set of instructions for using the HDACi and/or the complex protein composition.
14. The kit of any of items 7-13, wherein either one or both of the HDACi and the complex protein composition are in a concentrated form and require dilution prior to use.
15. The kit of any of items 7-14 further comprising one or more plant growth regulators selected from auxins, cytokinins, gibberellins, abscisic acid, and mixtures thereof.
16. The kit of items 15, wherein the one or more plant growth regulators can be comprised in the same container as the HDACi and/or the complex protein composition or in a separate container.
17. The kit of any of items 7-16 further comprising one or more chromosome doubling agent, such as colchicine, contained in a further container.
18. Use of a method according to any of items 1-6 or a kit according to any of items 7-17 for producing haploid, polyhaploid and/or doubled haploid plants of the species Beta vulgaris by androgenesis from isolated microspores.
19. Use of a HDACi and a complex protein composition for producing haploid, polyhaploid and/or doubled haploid embryos and/or plants of the species Beta vulgaris by androgenesis from isolated microspores.

While several possible embodiments are disclosed above, embodiments of the present invention are not so limited. These exemplary embodiments are not intended to be exhaustive or to unnecessarily limit the scope of the invention, but instead were chosen and described in order to explain the principles of the present invention so that others skilled in the art may practice the invention. Indeed, various modifications of the invention in addition to those described herein will become apparent to those skilled in the art from the foregoing description. Such modifications are intended to fall within the scope of the appended claims. Further, the terminology employed herein is used for the purpose of describing exemplary embodiments only and the terminology is not intended to be limiting since the scope of the various embodiments of the present invention will be limited only by the appended claims and equivalents thereof. The scope of the invention is therefore indicated by the following claims, rather than the foregoing description and above-discussed embodiments, and all changes that come within the meaning and range of equivalents thereof are intended to be embraced therein.

All patents, applications, publications, test methods, literature, and other materials cited herein are hereby incorporated by reference in their entirety as if physically present in this specification.

1. A method for producing a haploid, polyhaploid and/or doubled haploid embryo, callus, seed and/or plant of the species Beta vulgaris comprising:
i) providing at least one isolated microspore of Beta vulgaris,
ii) culturing the at least one microspore from step i) in the presence of a complex protein composition and at least one histone deacetylase inhibitor (HDACi),
iii) culturing the at least one microspore from step ii) to obtain a callus or an embryo, and
iv) optionally, regenerating and thereby obtaining at least one plant from the callus or embryo of step iii), and preferably obtaining at least one haploid, polyhaploid and/or doubled haploid seed thereof.

2. The method of claim 1, wherein step i) comprises one or more of the following steps:
(a) providing a bud or an inflorescence or a part thereof comprising microspores at the tetrad and uninuclear development stage,
(b) optionally, disinfecting the bud or an inflorescence or the part thereof of (a),
(c) transferring the bud or an inflorescence or the part thereof of (a) or (b) to medium containing macro- and microsalts, saccharides and a complex protein composition,
(d) homogenizing the medium containing the bud or an inflorescence or the part thereof of step (c) and thereby producing a cell suspension,
(e) optionally, purifying the cell suspension preferably by means of a strainer and/or centrifugation thereby
(f) producing a suspension of microspores with a density of around 10,000 to 500,000 microspores per ml medium,
(g) optionally, cultivating the microspore suspension in the dark, and
(h) adding the at least one histone deacetylase inhibitor (HDACi) to a final concentration of 1 nM to 10 μM, preferably 100 nM to 10 μM, more preferably 1 μM to 10 μM.

3. The method of claim 1, further comprising in the step ii) one or more of the following steps:
(i) optionally, cultivating the microspore suspension in the dark,
(j) adding the at least one histone deacetylase inhibitor (HDACi) to a final concentration of 1 nM to 10 μM, preferably 100 nM to 10 μM, more preferably 1 μM to 10 μM and
(k) removing the at least one histone deacetylase inhibitor (HDACi) from the microspore culture of step ii).

4. The method of claim 1, wherein the at least one histone deacetylase inhibitor (HDACi) is selected from the group consisting of trichostatin A (TSA), hydroxamic acids and hydroxamates, such as vorinostat (SAHA), belinostat (PXD101), dacinostat (LAQ824), and panobinostat (LBH589), cyclic tetrapeptides, such as trapoxin B and depsipeptides, such as romidepsin (FK228), benzamides such as entinostat (MS-275), tacedinaline (CI994), and mocetinostat (MGCD0103), electrophilic ketones, and aliphatic acid compounds such as phenylbutyrate and valproic acid.

5. The method of claim 1, wherein the complex protein composition comprises or consists of hydrolysed or partially hydrolysed protein matter derived from milk, such as casein or whey, animals, such as meat or fish, cereal, such as rice or corn, plants, such as soybean or combinations thereof; and/or the complex protein composition comprises or consists of hydrolysed milk protein isolates, hydrolysed lactoprotein concentrate, hydrolysed casein isolates, casein hydrolysates, hydrolysed lactalbumin, hydrolysed casein sodium, hydrolysed calcium caseinate, hydrolysed full cow's milk, partially or completely skimmed milk, hydrolysed soy protein isolate, hydrolysed soybean concentrate or combinations thereof; and/or the complex protein composition comprises or consists of a proteolysate selected from the group consisting of casein hydrolysate, soybean hydrolysate, rice proteolysate, potato protein hydrolysate, fish protein hydrolysate, ovalbumin hydrolysate, lactalbumin hydrolysate, glutin hydrolysate, animal and plant proteolysate and combinations thereof.

6. The method according to claim 1, wherein the at least one histone deacetylase inhibitor (HDACi) is present at a concentration from about 1 nM to about 10 μM, preferably 100 nM to 10 μM, more preferably 1 μM to 10 μM in the culture medium used in step ii) and/or wherein the complex protein composition is present at a concentration from about 100 to about 20,000 mg/l, preferably about 500 to about 15,000 mg/l, particularly preferably about 2,000 to about 10,000 mg/l in the culture medium used in step ii).

7. The method according to claim 1, wherein the callus or embryo is contacted with one or more plant growth regulator(s) selected from the group consisting of auxins, cytokinins, gibberellins, abscisic acid, and mixtures thereof in step iv).

8. The method according to claim 1, wherein one or more chromosome doubling agent(s), such as colchicine, is/are added during step iii) and/or step iv).

9. A kit for producing a haploid, polyhaploid and/or doubled haploid embryo, callus, seed and/or plant of the species Beta vulgaris from at least one isolated microspore comprising:
(a) at least one histone deacetylase inhibitor (HDACi), and
(b) a complex protein composition,
wherein the at least one histone deacetylase inhibitor (HDACi) and the complex protein composition are comprised within the same container or within two or more separate containers.

10. The kit of claim 9, wherein the at least one histone deacetylase inhibitor (HDACi) is selected from the group consisting of trichostatin A (TSA), hydroxamic acids and hydroxamates, such as vorinostat (SAHA), belinostat (PXD101), dacinostat (LAQ824), and panobinostat (LBH589), cyclic tetrapeptides, such as trapoxin B and depsipeptides, such as romidepsin (FK228), benzamides such as entinostat (MS-275), tacedinaline (C1994), and mocetinostat (MGCD0103), electrophilic ketones, and aliphatic acid compounds such as phenylbutyrate and valproic acid.

11. The kit of claim 9, wherein the complex protein composition comprises or consists of hydrolysed or partially hydrolysed protein matter derived from milk, such as casein or whey, animals, such as meat or fish, cereal, such as rice or corn, plants, such as soybean or combinations thereof; and/or the complex protein composition comprises or consists of hydrolysed milk protein isolates, hydrolysed lactoprotein concentrate, hydrolysed casein isolates, casein hydrolysates, hydrolysed lactalbumin, hydrolysed casein sodium, hydrolysed calcium caseinate, hydrolysed full cow's milk, partially or completely skimmed milk, hydrolysed soy protein isolate, hydrolysed soybean concentrate or combinations thereof; and/or the complex protein composition comprises or consists of a proteolysate selected from the group consisting of casein hydrolysate, soybean hydrolysate, rice proteolysate, potato protein hydrolysate, fish protein hydrolysate, ovalbumin hydrolysate, lactalbumin hydrolysate, glutin hydrolysate, animal and plant proteolysate and combinations thereof.

12. The kit of claim 9, wherein the kit further comprises one or more plant growth regulator(s) selected from auxins, cytokinins, gibberellins, abscisic acid, and mixtures thereof and/or wherein the kit further comprises one or more chromosome doubling agent(s) such as colchicine.

13. A method of using a histone deacetylase inhibitor (HDACi) as defined in claim 4 and a complex protein composition for producing a haploid, polyhaploid and/or doubled haploid embryo, callus and/or plant or seed of the species Beta vulgaris, the method comprising:
i) providing at least one isolated microspore of Beta vulgaris,
ii) culturing the at least one microspore from step i) in the presence of a complex protein composition and at least one histone deacetylase inhibitor (HDACi),
iii) culturing the at least one microspore from step ii) to obtain a callus or an embryo, and
iv) optionally, regenerating and thereby obtaining at least one plant from the callus or embryo of step iii), and preferably obtaining at least one haploid, polyhaploid and/or doubled haploid seed thereof,
wherein the complex protein composition comprises or consists of hydrolysed or partially hydrolysed protein matter derived from milk, such as casein or whey, animals, such as meat or fish, cereal, such as rice or corn, plants, such as soybean or combinations thereof; and/or the complex protein composition comprises or consists of hydrolysed milk protein isolates, hydrolysed lactoprotein concentrate, hydrolysed casein isolates, casein hydrolysates, hydrolysed lactalbumin, hydrolysed casein sodium, hydrolysed calcium caseinate, hydrolysed full cow's milk, partially or completely skimmed milk, hydrolysed soy protein isolate, hydrolysed soybean concentrate or combinations thereof; and/or the complex protein composition comprises or consists of a proteolysate selected from the group consisting of casein hydrolysate, soybean hydrolysate, rice proteolysate, potato protein hydrolysate, fish protein hydrolysate, ovalbumin hydrolysate, lactalbumin hydrolysate, glutin hydrolysate, animal and plant proteolysate and combinations thereof.

14. A population of haploid, polyhaploid and/or doubled haploid Beta vulgaris plants directly derived from a single flower, single inflorescence, or single bud, preferably obtained or obtainable by a method according to claim 1.

15. The population of haploid, polyhaploid and/or doubled haploid Beta vulgaris plants according to claim 14, wherein the population comprises at least 10 individuals.

16. The method according to claim 4, wherein the at least one histone deacetylase inhibitor (HDACi) is trichostatin A (TSA) or romidepsin (FK228).

17. The kit according to claim 10, wherein the at least one histone deacetylase inhibitor (HDACi) is trichostatin A (TSA) or romidepsin (FK228).

18. The method according to claim 5, wherein the hydrolysis degree is in a range from about 20 to about 80%, preferably from about 30 to about 80%, particularly preferably from about 40 to about 60%.

19. The kit according to claim 11, wherein the hydrolysis degree is in a range from about 20 to about 80%, preferably from about 30 to about 80%, particularly preferably from about 40 to about 60%.

20. The method according to claim 13, wherein the hydrolysis degree is in a range from about 20 to about 80%, preferably from about 30 to about 80%, particularly preferably from about 40 to about 60%.
